(19) United States
(12) Patent Application Publication
(10) Pub. No.: US 2025/0346929 A1
(43) Pub. Date: Nov. 13, 2025

(54) IMMOBILIZED ENZYMES FOR THE BIOELECTRIC PRODUCTION OF HYDROGEN PEROXIDE

(71) Applicant: Anodyne Chemistries, Inc., Burnaby (CA)

(72) Inventors: Emmanuelle DAVIES, Burnaby (CA); Robert GREENE, Burnaby (CA); Ching Ning SHEN, Burnaby (CA)

(21) Appl. No.: 19/275,299

(22) Filed: Jul. 21, 2025

Related U.S. Application Data
(63) Continuation of application No. PCT/CA2025/050644, filed on May 1, 2025.
(60) Provisional application No. 63/641,260, filed on May 1, 2024.

(51) Int. Cl. C12P 3/00; C12N 9/02; C12N 9/06; C12N 11/14

(52) U.S. Cl. CPC C12P 3/00; C12N 9/0026; C12N 9/0036; C12N 11/14; C12Y 105/05001; C12Y 106/03

(57) ABSTRACT

The present invention relates to enzymatic reactor cells and related methods of use, e.g., to produce a compound or product such as hydrogen peroxide by using an enzymatic reactor cell, wherein the enzymatic reactor cell includes a surface, a linker, and one or more enzymes.

### CROSS REFERENCE TO RELATED APPLICATIONS

This application is a Continuation Application of International Application No. PCT/CA2025/050644, filed May 1, 2025, claims priority to U.S. Provisional Application No. 63/641,260, filed May 1, 2024, the disclosure of which is herein incorporated by reference in their entirety for all purposes.

### REFERENCE TO AN ELECTRONIC SEQUENCE LISTING

The contents of the electronic sequence listing (ANOD_003_01US_SeqList_ST26.xml; Size: 171,563 bytes; and Date of Creation: Jul. 18, 2025) are herein incorporated by reference in its entirety.

### BACKGROUND OF THE INVENTION

Enzymatic bioelectrocatalysis is increasing in popularity for the production of various commodities such as methanol, or other forms of energy. Oxidoreductase enzymes are key enzymes in microorganisms and have been utilized or mutated to catalyze crucial redox reactions. Cofactors and/or electrodes can also be utilized to enable electron transfer from the active site of the enzyme and the substrate. The overall reaction pathway to create a single commodity, however, can be complex, as it can require multiple enzymes, cofactors and substrates, adding to the complexity of enzymatic bioelectrocatalysis to create a final product or commodity. Use of multiple such oxidoreductase enzymes and cofactors or other enzymes has been used for example in bioreactors or fermenters with limited success, as it has been difficult to manipulate and control the intended reactions. The use of such enzymatic bioelectrocatalysis to provide efficient and intended production of commodities needs improvement. Such ideas include the use of a reactor cell or multiple reactor cells wherein an enzyme is immobilized to a surface, and the reaction is supported by an electrode for controlled and efficient electron transfer in the reaction. Improvements in this field are still needed.

### SUMMARY OF THE INVENTION

The disclosure provides enzymatic reactor cells and related methods of use, e.g., to produce a compound or product e.g., hydrogen peroxide by using an enzymatic reactor cell. In a specific embodiment, the enzymatic reactor cell, includes a surface, a surface linker or electrode surface linker, and one or more enzymes, wherein the surface is directly linked to the surface or electrode surface linker and the surface linker is further directly linked to the enzyme. In a specific embodiment, the surface linker may be an electrode surface linker. In a specific embodiment, the enzymatic reactor cell has the following formula I:

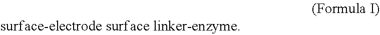

In a specific embodiment, Formula I can specifically be as follows:

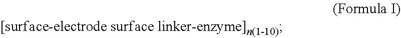

wherein when n is 2-10, each of the 2-10 [surface—electrode surface linker—enzyme] comprises a different surface, electrode surface linker and/or enzyme from the other [surface—electrode surface linker—enzyme].

In a specific embodiment, Formula II can specifically be as follows:

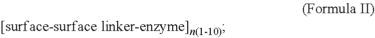

wherein when n is 2-10, each of the 2-10 [surface—surface linker—enzyme] comprises a different surface, surface linker and/or enzyme from the other [surface—electrode surface linker—enzyme].

In another specific embodiment, n of Formula I or Formula II is 1, 2, 3, 4, 5, 6, 7, 8, 9, or 10. In another specific embodiment, n of Formula I or Formula II is 11, 12, 13, 14, 15, 16, 17, 18, 19, or 20.

In another embodiment, the surface is conductive, thereby allowing electron transfer between the enzyme and the surface or electrode surface. In another specific embodiment the electron transfer travels from the enzyme to the surface. In another specific embodiment the electron transfer travels from the surface to the enzyme. In a specific embodiment, the enzyme is an oxidoreductase enzyme. In embodiments, the enzyme produces hydrogen peroxide.

In a specific embodiment, the electrode surface linker is conductive, thereby allowing electron transfer from the oxidoreductase enzyme and the electrode surface and comprises a surface binding moiety (SBM), and in a specific embodiment the electrode surface linker providing election transfer comprises a peptide, protein, a chemical polymer, or polynucleotide described herein.

In embodiments, the enzymatic reactor cell comprises an enzyme that produces hydrogen peroxide. In embodiments, the enzymatic reactor cell comprises an enzyme that produces hydrogen peroxide from oxygen, in parallel with another product e.g., glucose oxidase, alcohol oxidase, formate oxidase, sulfite oxidase, alditol oxidase, pyruvate oxidase, lactate oxidase, amine oxidase, or glycolate oxidase. In embodiments, the enzymatic reactor cell comprises an enzyme that produces hydrogen peroxide from oxygen e.g., methane monooxygenase reductase subunit, NAD(P)H oxidase. In embodiments, the enzymatic reactor cell comprises hydrogen Peroxide producing enzymes with one or more mutation(s) to introduce new functional groups near the surface of the enzyme (e.g., cysteine, lysine, etc.). In embodiments, the enzymatic reactor cell comprises hydrogen peroxide producing enzymes with one or more mutations to enhance the size, activity, or stability of the enzyme. In embodiments, the enzymatic reactor cell comprises hydrogen peroxide producing enzymes with an affinity based purification tag (e.g., polyhistidine tag, glutathione-S-transferase, FLAG tag, Streptavidin-binding tag, maltose-binding tag, etc.). In embodiments, the enzymatic reactor cell comprises methane monooxygenase reductase e.g., from Methylomonas methanica. In embodiments, the enzymatic reactor cell comprisesa NAD(P)H Oxidase e.g., from Thermus thermophilus. In an embodiment, the enzyme that produces hydrogen peroxide as described herein is immobilized on the electrode surface. In embodiments, the enzyme that produces hydrogen peroxide as described herein is not immobilized on the electrode surface.

In another embodiment, the enzymatic reactor cells, fusion proteins, linkers, or methods may be from PCT application PCT/US2023/078253, filed Oct. 30, 2023, which is incorporated by reference in its entirety herein.

### BRIEF DESCRIPTION OF THE DRAWINGS

Further features and advantages of the present disclosure will become apparent from the following detailed description, taken in combination with the appended drawings, in which:

FIG. 1 illustrates a reactor cell for producing hydrogen peroxide from oxygen with a hydrogen peroxide producing enzyme.

FIG. 2 illustrates a reactor cell for producing hydrogen peroxide from oxygen with a hydrogen peroxide producing enzyme wherein the oxygen at the anode is recirculated to act a reagent at the cathode.

FIG. 3 illustrates the production of H₂O₂ in μM over time when Enzyme (Thermus thermophilus HB27 NADH Oxidase)+Linker (includes MBP SEQ ID No. 104) fusion with MBP on the N terminus of the fusion protein is adsorbed to the Stainless steel (SS) disk surface in comparison to enzyme only or the SS surface only.

FIG. 4 illustrates the faradaic efficiency expressed as a percentage over time for the production of H₂O₂ when Enzyme (Thermus thermophilus HB27 NADH Oxidase)+Linker (includes MBP SEQ ID No. 104) with MBP on the N terminus is adsorbed to the SS disk surface in comparison to enzyme only and SS surface only

FIG. 5 illustrates the production of H₂O₂ in μM over time when Enzyme (Thermus thermophilus HB27 NADH Oxidase)+Linker (includes MBP SEQ ID NO. 104) fusion with MBP on the C terminus of the fusion protein is adsorbed to the Stainless steel (SS) disk surface in comparison to enzyme only or the SS surface only.

FIG. 6 illustrates the faradaic efficiency expressed as a percentage over time for the production of H₂O₂ when Enzyme (Thermus thermophilus HB27 NADH Oxidase)+Linker (includes MBP SEQ ID NO. 104) with MBP on the C terminus is adsorbed to the SS disk surface in comparison to enzyme only and SS surface only.

FIG. 7 illustrates the faradaic efficiency expressed as a percentage over time for the production of H₂O₂ when Enzyme (Thermus thermophilus HB27 NADH Oxidase)+Linker (SEQ ID No. 25 and GS nanowire) is adsorbed to the glassy carbon (GC) disk surface in comparison to enzyme only and GC surface only.

FIG. 8 shows the faradaic efficiency expressed as a percentage over time for the production of H₂O₂ when Enzyme (Thermus thermophilus HB27 NADH Oxidase)+Linker (SEQ ID No. 25 and GS nanowire) is adsorbed to the carbon paper (CP) surface in comparison to enzyme only and CP surface only.

### DETAILED DESCRIPTION

The following terms are defined below.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

Throughout the present specification, the terms “about” and/or “approximately” may be used in conjunction with numerical values and/or ranges. The term “about” is understood to mean those values near to a recited value. Furthermore, the phrases “less than about [a value]” or “greater than about [a value]” should be understood in view of the definition of the term “about” provided herein. The terms “about” and “approximately” may be used interchangeably.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range may be an endpoint for the range encompassed thereby (e.g., the range 50-80 includes the ranges with endpoints such as 55-80, 50-75, etc.).

The term “a” or “an” refers to one or more of that entity; the terms “a” (or “an”), “one or more” and “at least one” are used interchangeably herein. In addition, reference to “an inhibitor” by the indefinite article “a” or “an” does not exclude the possibility that more than one of the inhibitors is present, unless the context clearly requires that there is one and only one of the inhibitors. As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “a linker sequence” is intended to mean a sequence that bridges the surface binding entity, e.g., inorganic surface entity, with the organic binding entity, such as an enzyme. As used herein, a linker sequence may comprise one or both of an active linker and/or a passive linker. Thus, a linker sequence may, for example, comprise the amino acid sequence of protein G from Streptococcus or streptavidin from Streptomyce, or may be a simple amino acid sequence or simply a single bond, such as a covalent bond. The linkage can also be a non-covalent bond to the enzyme and/or surface. Organic binding entities include both synthetic carbon-based compounds as well as biologically-derived molecules. The linker may include additional functional features, such as being a cofactor for an enzyme, including the enzyme directly linked to the linker.

The term “surface binding motif” or SBM is intended to mean a molecule with specific and selective affinity for an organic or inorganic substance, such as, e.g., gold, silica, silver, plastic, polystyrene, cellulose (e.g., nitrocellulose), and graphene, graphite, carbon paper, carbon felt, SWCNT, MWCNT, carbon black etc, copper, platinum, palladium, nickel, iron, zinc, titanium, aluminium, zinc, stainless Steel, alloys etc, metal oxide e.g., zinc oxide, iron oxide, titanium oxide, tin oxide, aluminium oxide etc., cellulose, polystyrene, PDMS, chlorine doped polypyrrole, polypropylene etc., silica, silicon etc. An SBM may be a peptide or polypeptide.

The term “covalent fusion” is intended to mean the joining of two or more genes that encode separate peptides or proteins. The terms “polypeptide”, “protein” and “peptide” are used interchangeably and mean a polymer of amino acids not limited to any particular length. The term does not exclude modifications such as myristylation, sulfation, glycosylation, phosphorylation and addition or deletion of signal sequences. The terms “polypeptide” or “protein” or “peptide” means one or more chains of amino acids, wherein each chain comprises amino acids covalently linked by peptide bonds, and wherein said polypeptide or protein or peptide can comprise a plurality of chains non-covalently and/or covalently linked together by peptide bonds, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. Thus, a “polypeptide” or a “protein” can comprise one (termed “a monomer”) or a plurality (termed “a multimer”) of amino acid chains.

The term “fusion protein” means a protein comprised of at least two different amino acid sequences and generated within an organism such as E. coli. An inorganic surface binding peptide expressed with an A or G protein or a linker is an example of a fusion protein.

As used herein, the “alignment” of two or more protein/amino acid sequences may be performed using the alignment at program Clustal W2, available www.ebi.ac.uk/Tools/msa/clustalw2/. The following default parameters may be used for Pairwise alignment: Protein Weight Matrix=Gonnet; Gap Open=10; Gap Extension=0.1. Any sequence alignment or determination of sequence identity of proteins or amino acid sequences is determined by the software or alignment program described herein.

The term “specifically binds” means that a molecule reacts or associates more frequently, more rapidly, with greater duration and/or with greater affinity with a particular target molecule, e.g., a pathogen or surface, than it does with alternative molecules, e.g., pathogens or other surfaces. It is also understood by reading this definition that, a molecule that specifically or preferentially binds to a first target may or may not specifically or preferentially bind to a second target. As such, “specific binding” does not necessarily require (although it can include) exclusive binding.

Features and advantages of the subject matter hereof will become more apparent in light of the following detailed description of selected embodiments, as illustrated in the accompanying figures. As will be realized, the subject matter disclosed and claimed is capable of modifications in various respects, all without departing from the scope of the claims. Accordingly, the drawings and the description are to be regarded as illustrative in nature, and not as restrictive and the full scope of the subject matter is set forth in the claims.

In this disclosure, the word “comprising” is used in a non-limiting sense to mean that items following the word are included, but items not specifically mentioned are not excluded.

It will be understood that in embodiments which comprise or may comprise a specified feature or variable or parameter, alternative embodiments may consist, or consist essentially of such features, or variables or parameters. A reference to an element by the indefinite article “a” does not exclude the possibility that more than one of the elements is present, unless the context clearly requires that there be one and only one of the elements.

In this disclosure the recitation of numerical ranges by endpoints includes all numbers subsumed within that range including all whole numbers, all integers and all fractional intermediates (e.g., 1 to 5 includes 1, 1.5, 2, 2.75, 3, 3.80, 4, and 5, etc). In this disclosure the singular forms an “an”, and “the” include plural referents unless the content clearly dictates otherwise. Thus, for example, reference to a composition containing “a compound” includes a mixture of two or more compounds.

In this disclosure term “or” is generally employed in its sense including “and/or” unless the content clearly dictates otherwise.

### Embodiments of the Invention

Bioelectrocatalysis includes reactions that are catalyzed by biologically active materials in association with electrically conductive electrodes.

Enzymatic bioelectrocatalysis is a specific form of bioelectrocatalysis or electrocatalysis using an enzyme for catalyzing a certain reaction. In one general example of enzymatic bioelectrocatalysis, enzymes are associated with an electrode, including electrode linkers, in a manner that allows electron transfer between the electrode and the enzymes. Such electron transfer allows the continued function of each enzyme over many catalyzed reactions, including a series of reactions to obtain a final desired compound or product. The term “enzyme(s)” as used herein thus relates to a biologically based catalytic mechanism, and can comprise a protein that is both wild-type or mutated for any intended reaction by the user. Nonlimiting examples of other biologically based catalytic materials can include eukaryotic cells, prokaryotic cells, cellular organelles, nucleic acid enzymes (i.e. deoxyribozymes), and the like.

Oxidoreductase enzymes are specific biocatalytic proteins that can catalyse the coupled oxidation and reduction with a substrate and/or cofactor, thus, transferring an electron(s) with the involvement of an electrode linker and/or a cofactor of the enzyme. In a specific embodiment of the present invention, the oxidoreductase enzyme can be used in a single reaction or in a series of reactions with other enzymes or oxidoreductase enzymes wherein the oxidoreductase enzyme is directly linked to, for example, an electrode linker. In a specific embodiment, the enzymes or oxidoreductase enzyme is directly linked to an electrode linker in a device, biodevice or reactor cell that contains the reaction or series of reactions if more than one enzyme or oxidoreductase enzyme is used.

In a specific embodiment, the device, biodevice or reactor cell allows for the immobilization of an enzyme or oxidoreductase enzymes. In a specific embodiment, the device, biodevice or reactor cell comprises a surface that allows for immobilization of the enzyme or oxidoreductase enzyme. In another embodiment, the surface may be an electrode surface. This surface may include conductive or non-conductive material.

In another embodiment, the surface may have active groups or other means for attaching an enzyme or other means for linking the enzyme to the surface. In one specific embodiment, a linker is specifically used to link to the surface (such as covalently or non-covalently) and then link to the enzyme. In a specific embodiment, the linker is covalently linked to the surface and covalently linked to the enzyme. In another embodiment, the linker can be covalently linked to just the surface or the enzyme. In another embodiment, the linker may be linked to the surface or enzyme by non-covalent means.

In one embodiment, the enzymatic process can utilise more than one device, biodevice or reactor cell. In one embodiment, several enzymatic reactor cells are constructed in series or in parallel. In another embodiment, several enzymatic reactor cells are constructed in series or in parallel each allowing for a different catalytic reaction. In a specific embodiment, a series of reactor cells can be used to comprise more than one enzymatic reactions to carry out a an enzymatic pathway, i.e., there is more than one enzyme used to achieve an end product or compound from a starting compound.

In one embodiment, the enzymatic bioelectrocatalysis is performed in an enzymatic reactor cell.

In a specific embodiment, the enzymatic reactor cell, comprises a surface, such as an electrode surface, or non-electrode surface; a linker or electrode surface linker; and one or more enzymes, wherein the electrode surface is linked to the electrode surface linker and the surface linker is further directly linked to the oxidoreductase enzyme. In a specific embodiment, the enzyme may be an any enzyme that produces hydrogen peroxide, either as the primary reaction or product, or as a second or minor reaction or product. In a specific embodiment, the enzyme produces hydrogen peroxide from oxygen. In another embodiment, the enzyme that produces hydrogen peroxide may also produce another product in parallel or in a sequence such as for example, the use of the enzyme glucose oxidase, alcohol oxidase, formate oxidase, sulfite oxidase, alditol oxidase, pyruvate oxidase, lactate oxidase, amine oxidase, glycolate oxidase or combinations thereof. In a specific embodiment, the parallel use of another enzyme may produce hydrogen peroxide from oxygen. In another embodiment, the enzyme that produces hydrogen peroxide may do so without another enzyme, including no other enzyme in parallel or in a sequential reaction. In a specific embodiment, the enzyme that produces hydrogen peroxide from oxygen is methane monooxygenase reductase subunit, and/or NAD(P)H oxidase.

In a specific embodiment, the enzyme that produces hydrogen peroxide may be a mutant enzyme. In a specific embodiment, the mutations in the enzyme may produce new functional groups near the surface of the enzyme, such as an amino acid mutated to a cysteine or a lysine. In another specific embodiment, the mutations may enhance the size, intended activity, or stability of the hydrogen peroxide producing enzyme.

In a specific embodiment, the enzyme is a methane monooxygenase reductase. In a specific embodiment, the methane monooxygenase reductase is from Methylomonas methanica. In another specific embodiment, the enzyme is a NADH Oxidase or NAD(P)H Oxidase. In a specific embodiment, the NADH Oxidase or NAD(P)H Oxidase is from Thermus thermophilus. In another specific embodiment, the enzyme that produces hydrogen peroxide may also produce superoxide, i.e., O₂⁻. In a specific embodiment, the superoxide forms into hydrogen peroxide.

In a specific embodiment, the source of oxygen for producing hydrogen peroxide may be from one or multiple sources. In one embodiment, the oxygen source is oxygen gas, dissolved oxygen, from air or specifically, compressed air, or combinations thereof. In another specific embodiment, the oxygen is at the anode of the enzymatic reactor cell and is recirculated as a reagent at the cathode.

In a specific embodiment, the enzymatic reactor cell has the following formula I:

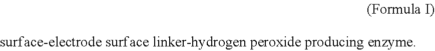

In a specific embodiment, Formula I can specifically be as follows:

[surface—electrode surface linker—hydrogen peroxide producing enzyme]ₙ₍₁₋₁₀₎ (Formula I);

wherein when n is 2-10, each of the 2-10 [surface—electrode surface linker—enzyme] comprises a different surface, electrode surface linker and/or enzyme from the other [surface—electrode surface linker—enzyme]. In a specific embodiment, at least one of n comprises an enzyme that is a mutant or wildtype form of a hydrogen peroxide producing enzyme. In a specific embodiment, the enzymes are methane monooxygenase reductase subunit, NADH oxidase and/or NAD(P)H oxidase.

In a specific embodiment, Formula II can specifically be as follows:

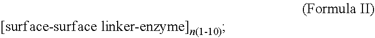

wherein when n is 2-10, each of the 2-10 [surface—surface linker—enzyme] comprises a different surface, surface linker and/or enzyme from the other [surface—electrode surface linker—enzyme]. In a specific embodiment, at least one of n comprises an enzyme that is a mutant or wildtype form of a hydrogen peroxide producing enzyme. In a specific embodiment, the enzymes are methane monooxygenase reductase subunit and/or NAD(P)H oxidase.

In another specific embodiment, n of Formula I or Formula II is 1, 2, 3, 4, 5, 6, 7, 8, 9, or 10. In another specific embodiment, n of Formula I or Formula II is 11, 12, 13, 14, 15, 16, 17, 18, 19, or 20.

In other words, the reactor cells or a single reactor cell, can include multiple types of surfaces or multiple types of linker or electrode surface linkers, and/or multiple enzymes. For example, a single reactor cell can include more than one enzyme from an enzymatic pathway, wherein the different enzymes in the pathway are attached to the surface of the reactor cell. In a specific embodiment, the different enzymes use different electrode surface linkers and/or different surfaces for a specific linkage for each enzyme, thereby partitioning or separating the different enzymes in the reactor cell as needed. In a specific embodiment, at least one of the reactions or reactor cells comprises a mutant or wildtype form of a hydrogen peroxide producing enzyme and produces hydrogen peroxide.

In a specific embodiment, the surface is attached or immobilized to the reactor cell. In another embodiment, the surface is free from and not attached to the reactor cell, such as being free flowing in a solution in the reactor cell.

In a specific embodiment, the surface or electrode surface is planar, non-planar, spherical, in the shape of nanoparticles, an aerogel, fibrous, or incorporated in a polymer. In a specific embodiment, the surface is a planar surface.

In a specific embodiment, the reactor call may have at least one surface or electrode surface that is an organic or inorganic surface and can be selected based on various advantages, including conductivity, ease of use in a reactor cell, and ease of attachment of the enzyme with the use of a linker. In a specific embodiment, the reactor cell may include more than one type of surface such as a planar surface at one end of the reactor cell and the use of beads in a different portion of the reactor cell.

In another embodiment, the surface or electrode surface of the reactor cell comprises a metal, a polymer, or a biological surface. In a specific embodiment, the reactor surface can include at least one surface or electrode surface that comprises one or more selected from the group consisting of graphite, platinum, zinc oxide, cellulose, polystyrene, gold, gold alloy, gold palladium alloy, silver, copper, nickel, iron, zinc, titanium, aluminium, stainless steel, metal alloys, and metal oxides such as iron oxide, titanium oxide, manganese oxide, etc. In embodiments, the reactor surface can include at least one surface or electrode surface that comprises one or more selected from the group consisting of graphite, platinum, cellulose, polystyrene, gold, gold palladium alloy, silver, copper, nickel, iron, zinc, titanium, aluminium, stainless steel, and metal oxides such as iron oxide, titanium oxide, manganese oxide, zinc oxide, etc. In another specific embodiment, the reactor surface can include at least one surface or electrode surface that comprises one or more selected from the group consisting of various carbon surfaces such as graphite, graphene, carbon paper, or carbon felt, teflonated carbon paper, teflonated carbon felt, carbon based gas diffusion electrode containing a macroporous and/or microporous layer, SWCNT, MWCNT, carbon black, activated carbon, graphite foil, platinum, zinc oxide, cellulose, polystyrene, gold, gold alloy, gold palladium alloy, and metal oxides such as iron oxide, titanium, titanium oxide, manganese oxide, aluminium oxide, and tin oxide, inorganic surfaces such as silica, silicon etc. In embodiments, the surface may be a polymer such as cellulose, polystyrene, PDMS, chlorine doped polypyrrole, polypropylene etc. In embodiments, the surface is an alloy or combination of two or more surface materials disclosed herein.

In another embodiment, the surface may be carbon based. In another specific embodiment, the carbon based surface may be Glassy carbon; carbon/graphite felt; Carbon nanofibers; Carbon nanotubes; Carbon black-coated graphite felt; Microporous carbon; Hierarchically porous carbon; and/or Mesoporous carbon.

In another embodiment, the material of the surface may relate to or include heteroatom doped carbon. In a specific embodiment, the heteroatom doped carbon may be or include Oxygen-doped carbon such as Oxidized graphite felt, —COOH doped carbon, C—O—C doped carbon, Oxygenated CNTs, Quinone incorporated carbon nanostructures, and/or Edge oxygenated graphitic nanoplatelets etc. In a specific embodiment, the heteroatom doped carbon may include Nitrogen-doped carbon such as Nitrogen-rich layered graphene. In a specific embodiment, the heteroatom doped carbon may include Boron-doped carbon, Sulphur-doped carbon, and/or Fluorine-doped carbon.

In another embodiment, the material of the surface may relate to or include transition metal+carbon. In a specific embodiment, the transition metal+carbon may be or include Mesoporous carbon sphere doped with MnO and Mn—Nₓ moieties; Cobalt and nitrogen co-doped CNTs; Transition metal single-atom catalyst such as Carbon-supported nickel (Ni (II)) single-atom catalyst or Cobalt single-atom catalyst anchored in nitrogen-doped graphene; Nitrogen-doped carbon materials featuring atomically dispersed metal cations (M—N—C), including Nitrogen-doped carbon with cobalt dispersed surface (Co—N—C), or Cobalt-N₄ (Co—N₄) supported by graphene; Iron anchored to CNTs with neighboring oxygen coordination pathways (Fe—C—O); Penta nitrogen coordinated cobalt single atom catalyst with oxygen-doped carbon black (Co—N₅—C); Cobalt single-site catalyst with oxygen-modified Co-(pyrrolic N)₄ (CoPc-OCNT); and/or Fe/Co/Zn-tri-metal co-doped carbon nanofibers (Fe/Co/Zn@C-NCNFs-800).

In another embodiment, the material of the surface may relate to or include noble metal-based electrocatalysts. In a specific embodiment, the or include Silver-palladium (Au—Pd), noble metal-based electrocatalysts may be or include Platinum-mercury (Pt—Hg), and/or Palladium-mercury (Pd—Hg).

In another embodiment, the material of the surface may relate to or include carbon-free catalysts. In a specific embodiment, the carbon0free catalyst may be Ni₂Mo₆S₈; CuCo₂₋ₓNiₓS₄ (0≤x≤1.2); Perovskite oxide ceramic, Pb(NiWMnNbZrTi)_{1/6}O₃; m-BizO₃/TazO₅ electro-catalyst; and/or Ruddlesden-Popper perovskite oxide (Pr₂NiO_{4+δ}).

Depending on the need of use, such as multiple enzymatic steps and pathways, multiple surfaces can be used, for example to attach to linkers that are specific to that surface. Those specific linkers can then also be attached to a specific enzyme. The attachment of the linkers and thus the specific enzymes can then be partitioned based on the surface in the reactor cell.

In a specific embodiment of the reactor cell, the reactor cell may include at least one form of surface attached to an electrode surface linker that is conductive, thereby allowing electron transfer between the enzyme and the surface or electrode surface. In another specific embodiment the electron transfer travels from the enzyme to the surface. In another specific embodiment the electron transfer travels from the surface to the enzyme. In a specific embodiment, the enzyme is an oxidoreductase enzyme. In a particular embodiment, the enzyme produces hydrogen peroxide. In a specific embodiment, the enzymes are methane monooxygenase reductase subunit and/or NAD(P)H oxidase.

In a specific embodiment, the enzymatic reactor cells can utilize any form of linker as described herein, including a linker that includes a surface binding moiety (SBM) or a material binding peptide, a peptide, protein, a chemical, or polynucleotide. In a specific embodiment, the reactor cells utilize linkers that include a peptide or protein or a protein or peptide nanowire base. In a specific embodiment, these proteins or peptides are conductive and allow for electron transfer between an enzyme and the surface or other intended target in the reactor cell. In a specific embodiment, the linker that allows for electron transfer from the enzyme (such as an oxidoreductase enzyme) and the surface or electrode surface. In a specific embodiment the linkers are electrode surface linkers.

In one embodiment, the use of a peptide or protein in the linker thus allows fusion of the protein, i.e., the linker and the enzyme is a fusion protein, and can be expressed together, thereby allowing a single step of immobilizing the fusion protein to the intended surface.

In a specific embodiment, the reactor cells may comprise cells that allow for linkers bind to the surface and or the enzyme by covalent binding, cross-linking and/or entrapment. In a specific embodiment, the linker attaches by continuous protein expression, click chemistry, dithiol bond formation, Michael addition, a metal-sulfur bond linkage, nucleophilic substitution, a metal-nitrogen bond linkage, a metal-oxygen bond linkage, enzyme catalyzed conjugation, or EDC coupling to the surface and/or the enzyme. In one embodiment, the linker may comprise a chemical nanowire or DNA nanowire type base. In a specific embodiment, the chemical nanowire base can include polymer containing compounds (e.g., MAL-PEG-NHS), cyclic carbon containing compounds (e.g., Succinimidyl 4-(N-maleimidomethyl)cyclohexane-1-carboxylate), or dithiol containing compounds (e.g., 1,6-hexanedithiol). In another specific embodiment, the linker may use, be or include glutaraldehyde nanowires. In another specific embodiment, the linkers or nanowires may, use, be or include SMCC Succinimidyl 4-(N-maleimidomethyl)cyclohexane-1-carboxylate, bis-MAL-dPEG₃, TFP-dPEG₂Mal, MBS (m-maleimidobenzoyl-N-hydroxysuccinimide ester) or a maleimide.

In embodiments, the linker may comprise a nanowire which comprises a maleimide functional group, a tetrafluorophenyl functional group, an aldehyde functional group, an amine functional group, a N-hydroxysuccinimide functional group, a thiol functional group, a haloacetyl functional group, a pyridyl disulfide functional group, an imidoester functional group, an epoxide functional group, a hydrocarbon chain, one of more polyethylene glycol units, or one or more aromatic rings or a derivative thereof. For example, the nanowire may comprise maleimide, TFP-PEGₓ-Mal, glutaraldehyde, MBS (m-maleimidobenzoyl-N-hydroxysuccinimide ester), bis-MAL-PEGₓ, C-1,4-phenylene-bis-maleimide, 4,4′-Bis(maleoylamino)azobenzene/ethylene diamine, 6-aminocaproic sulfosuccinimidyl (4-acid, N,N-ethylene-bis(iodoacetamide), iodoacetyl)aminobenzoate, 2-(2-pyridinyldithio) ethaneamine, dimethyl pimelimidate, 1,4-Butanediol diglycidyl ether, dithiobismaleimidoethane, or 2-iminothiolane.

In embodiments, the SBM binds to a portion of the linker, the enzyme, and/or the surface by a chemical means or by a nanowire which uses or includes maleimide, TFP-PEGx-Mal, glutaraldehyde, MBS (m-maleimidobenzoyl-N-hydroxysuccinimide ester), bis-MAL-PEGx, C-1,4-phenylene-bis-maleimide, 4,4′-Bis(maleoylamino)azobenzene, ethylene diamine, 6-aminocaproic acid, N,N-ethylene-bis(iodoacetamide), sulfosuccinimidyl (4-iodoacetyl)aminobenzoate, 2-(2-Pyridinyldithio) ethaneamine, dimethyl pimelimidate, 1,4-Butanediol diglycidyl ether, dithiobismaleimidoethane, or 2-iminothiolane.

In a specific embodiment, the nanowire is a PEG based nanowire. In another embodiment, nanowire uses PEG with dithiol such as 1,6-hexanedithiol.

In one embodiment, the linker links by reversible mobilization, wherein the reversible mobilization includes binding by physisorption, bioaffinity, a metal-sulfur bond linkage, and chelation/metal binding. In a specific embodiment, the bioaffinity reversible mobilization is by biotin/streptavidin, a GST glutathione-S-transferase tag, a FLAG tag, a Streptavidin-binding tag, maltose-binding tag, or a His-tag. In other words, the reactor cells, can utilize protein/peptide technology to link to either the surface either covalently or non-covalently such as by high affinity binding moieties that are specific to that surface, or to the enzyme, either covalently (such as a fusion protein) or non-covalently such as by high affinity binding moieties that are specific to that enzyme, or a tag on that enzyme, such as a protein G/antibody or streptavidin/biotin system.

As described herein, the enzymatic reactor cells of the present invention can include one or more enzymes, including oxidoreductase enzymes. The reactor cells may be used for single reaction, using a single enzyme. The reactor cells can also be used for multiple reactions, using multiple enzymes in an enzymatic pathway. In a specific embodiment, the pathway can be performed in single reactor cell or more than one reactor cell may be used for the pathway, wherein different enzymes are used in different reactor cells.

Accordingly, one embodiment of the present invention is a system with more than one reactor cells, wherein each reactor cell comprises a different enzymatic reaction or the same enzymatic reaction. In a specific embodiment, the reactor cell comprises one oxidoreductase enzyme and performs a single enzymatic reaction in said enzymatic reactor cell.

In another embodiment, the enzymatic reactor cell comprises more than one oxidoreductase enzyme and performs more than one enzymatic reaction in said enzymatic reactor cell. In another embodiment, the more than one enzymatic reaction in said enzymatic reactor cell is a multi-step pathway. In another specific embodiment, the multi-step pathway is a biological pathway.

In a specific embodiment, the enzyme produces hydrogen peroxide from oxygen. In another embodiment, the enzyme that produces hydrogen peroxide may produce another product in parallel or in a sequence, such as for example, the use of the enzyme glucose oxidase, alcohol oxidase, formate oxidase, sulfite oxidase, alditol oxidase, pyruvate oxidase, lactate oxidase, amine oxidase, glycolate oxidase or combinations thereof. In a specific embodiment, the parallel use of another enzyme may produce hydrogen peroxide from oxygen. In another embodiment, the enzyme that produces hydrogen peroxide may do so without another enzyme, including no other enzyme in parallel or in a sequential reaction. In a specific embodiment, the enzyme that produces hydrogen peroxide from oxygen is methane monooxygenase reductase subunit, and/or NAD(P)H oxidase.

In a specific embodiment, the enzyme that produces hydrogen peroxide may be a mutant enzyme. In a specific embodiment, the mutations in the enzyme may produce new functional groups near the surface of the enzyme, such as an amino acid mutated to a cysteine or a lysine. In another specific embodiment, the mutations may enhance the size, intended activity, or stability of the hydrogen peroxide producing enzyme.

In a specific embodiment, the enzyme is a methane monooxygenase reductase. In a specific embodiment, the methane monooxygenase reductase is from Methylomonas methanica. In another specific embodiment, the enzyme is a NAD(P)H Oxidase. In a specific embodiment, the NAD(P)H Oxidase is from Thermus thermophilus. In another specific embodiment, the enzyme that produces hydrogen peroxide may also produce superoxide, i.e., O₂⁻. In a specific embodiment, the superoxide forms into hydrogen peroxide.

In another embodiment, the reactor cells comprise enzymes, such as enzymes that produce hydrogen peroxide that are not attached to any surface. In a specific embodiment, the reactor cells may have one or more enzymes that are attached to the surface and another set of enzymes (such as different enzymes in that pathway) that are not attached to the surface. In a specific embodiment, the oxidoreductase enzyme is linked to the electrode surface by the electrode surface linker, and one or more enzymes that are not linked to the surface.

In embodiment of the reactor cells of the present invention, the electrode surface is the cathode, the anode or both. In another specific embodiment, the reactor cells can utilize different electrodes known in the art. For example, the electrode can be made from a carbon-based material such as, for example, CNTs, carbon infiltrated CNTs (CI-CNTs), SWCNTs, MWCNTs, graphene, carbon black, carbon felt, carbon powder, carbon fiber, carbon paper (Toray, ELAT, etc.), graphite, teflonated carbon paper, teflonated carbon felt, carbon based gas diffusion electrode containing a macroporous and/or microporous layer, activated carbon, graphite foil, pyrolytic carbon, carbon cloth, screen printed carbon, doped diamond, doped diamond-like carbon (DLC), doped polycrystalline diamond (PCD), graphene-coated diamond, DLC, or PCD, and the like.

Electrodes can additionally be made from semiconductive materials, and any semiconductor material capable of electron transport/tunneling to and from the bioelectric material is considered to be within the present scope. Nonlimiting examples of semiconductor materials can include silicon, germanium, which can be doped with elements such as antimony, arsenic, boron, indium, gallium, phosphorus, or combinations thereof.

Electrodes made from conductive metals can also be used in the embodiments of the present disclosure. Any metal or metal alloy material that is electrically conductive, that can support a bioelectric material as described herein, and is capable of enzymes immobilized with a linked or adjacent redox linker or polymer.

Additionally, the present electrodes can be made from metal oxides, metal sulfides, and the like. Any oxide material that is electrically conductive, that can support a bioelectric material as described herein, and is capable of enzymes immobilized in with a linked or adjacent redox linker or polymer is considered to be within the present scope. Nonlimiting examples of oxides can include boron nitride materials, cerium oxide materials, indium-tin oxide (ITO), molybdenum sulfide, titanium oxides, including nanoporous titanium oxide, tin oxides, including tin oxide coated glass, and the like, including combinations thereof. In another embodiment, the reactor cells can use an electrode such as a 3-electrode cell, a rotating disk electrode, a rotating ring disk electrode, a through plane conductivity test set up, or other standard electrochemistry set up. In another specific embodiment, the reactor cell is an electrolyser flow cell such as with a gas diffusion electrode, a flow cell stack, and/or a flow cell with a gas diffusion electrode, or an electrolyser flow cell with gas diffusion electrode, electrolyser flow cell stack, or electrolyser flow cell stack with gas diffusion electrode.

In a specific embodiment, the reactor cell of the present invention includes an enzyme and/or oxidoreductase enzyme that produces hydrogen peroxide.

In another specific embodiment, the enzyme and/or oxidoreductase enzyme are an enzyme and/or oxidoreductase enzymes from a pathway that produces hydrogen peroxide.

In another embodiment, the reactor cell may be as depicted in Example 1. In another specific embodiment, the reactor cell or reactor cells may be primed or introduced with a starting material, substrate or any cofactors needed for the enzymatic reaction or pathway to proceed. In a specific embodiment, the reactor cells may initially comprise oxygen.

In another embodiment, the reactor cells are primed or introduced with other reagents in a reaction.

In a specific embodiment, one reactor cell comprises one oxidoreductase enzyme and performs a single enzymatic reaction in said enzymatic reactor cell.

In one embodiment of the present invention is a system with more than one reactor cell, wherein each reactor cell comprises a different enzymatic reaction or the same enzymatic reaction. In a specific embodiment, the plurality of the enzymatic reactor cells are in a series or are in parallel. Accordingly, the present invention may include a plurality of enzymatic reactor cells described herein.

The present invention also includes methods of using the reactor cells and enzymatic pathways described herein. In a specific embodiment, the methods include the use of a reactor cell as described herein to produce hydrogen peroxide.

### Surfaces of the Present Invention

Surfaces of the present invention may include an electrode or non-electrode surface depending on the needs to the enzymatic reaction or enzymatic pathway. In a specific embodiment, the surface is an electrode surface, and can function as either the anode, the cathode, or both in the enzymatic reactor cell. In a specific embodiment, the surface may include graphite, platinum, zinc oxide, cellulose (such as nitrocellulose), polystyrene, gold, gold alloy, gold palladium alloy, silver, copper, nickel, iron, zinc, titanium, aluminium, stainless steel, metal alloys, and metal oxides such as iron oxide, titanium oxide, manganese oxide, etc., or any combination thereof. In another specific embodiment, the surface may include graphite, platinum, zinc oxide, cellulose (such as nitrocellulose, microcrystalline cellulose, or paper), polystyrene, gold, gold alloy, gold palladium alloy, and metal oxides such as iron oxide, titanium, titanium oxide, manganese oxide, zinc oxide, aluminium oxide, tin oxide, silica, silica coated zinc oxide, etc., or any combination thereof. In another specific embodiment, the reactor surface can include at least one surface or electrode surface that comprises one or more selected from the group consisting of various carbon surfaces such as graphite, graphene, carbon paper, or carbon felt, teflonated carbon paper, teflonated carbon felt, carbon based gas diffusion electrode containing a macroporous and/or microporous layer, SWCNT, MWCNT, carbon black, activated carbon, graphite foil, platinum, zinc oxide, cellulose, polystyrene, gold, gold alloy, gold palladium alloy, and metal oxides such as iron oxide, titanium, titanium oxide, manganese oxide, zinc oxide, aluminium oxide, tin oxide, and inorganic surfaces such as silica, silicon etc. In another specific embodiment, the surface may be titanium, cellulose, polystyrene, carbon surfaces, and/or silica. In embodiments, the surface may be a polymer such as cellulose, polystyrene, PDMS, chlorine doped polypyrrole, polypropylene etc. In a specific embodiment, the surface may allow for a specific binding peptide to bind, wherein the binding peptide is the linker or a portion of the linker. In a specific embodiment, the surface may be for example gold, cellulose, or polystyrene, thereby allowing for affinity binding to a specific binding motif, or a covalent bond to the surface. In embodiments, the surface is an alloy or combination of two or more surface materials disclosed herein.

In another embodiment, the surfaces may be in various shapes or forms to facilitate immobilization of the enzyme and/or the reaction as desired. For example, the surface may be planar, non-planar, spherical, in the shape of nanoparticles, in the shape of beads, an aerogel, fibrous, and/or combinations of these forms, or be or include a polymer. In a specific embodiment, the surface may be planar allowing for one or more types of enzymes to be immobilized to the surface. In a specific embodiment, the surface may be in the form of nanoparticles. In another embodiment, the nanoparticles may be silica coated zinc oxide nanoparticles.

### Linkers of the Present Invention

Linkers may be included in the present invention. In one or more embodiment, the linkers of the present invention may have one or more of the following features: 1) a means for linking to the surface; 2) a means for linking to the enzyme, thereby providing a link between the surface and the enzyme; and 3) the linker is, or comprises a portion of the linker that allows for electron transfer between the enzyme (such as an oxidoreductase enzyme) and the surface or electrode surface. In a specific embodiment, the linker allows for electron transfer from the enzyme to the surface or from the surface to the enzyme.

In a specific embodiment the linkers are electrode surface linkers and are conductive. In a specific embodiment, the electrode surface linkers allow for electron transfer from the oxidoreductase enzyme and the surface or electrode surface. In one embodiment, the linker binds on one end to the surface, and the other end to the enzyme, thereby immobilizing the enzyme to the surface. In a specific embodiment, the linker is an electrode surface linker, thereby allowing electron transfer from the enzyme to the surface, or vice-versa. In a specific embodiment, the electrode surface linker is conductive, thereby allowing electron transfer from the oxidoreductase enzyme and the electrode surface.

In one embodiment, the linker or electrode surface linker binds to the surface.

In one embodiment, the linker or electrode surface linker comprises a surface binding moiety (SBM). In a specific embodiment, the surface binding moiety provide a means for binding to the surface, which may include various methods, including irreversible and reversible immobilization of the linker and enzyme to the surface. Irreversible immobilization includes covalent binding, cross-linking and entrapment, while reversible methods include physisorption, bioaffinity (biotin/streptavidin and protein A/G), chelation/metal binding, a metal-sulfur bond linkage, and disulfide bonds (LIEBANA; DRAGO, 2016). In embodiments, the surface binding moiety (SBM) binds to the surface by high affinity, non-covalent, physisorption binding.

In a specific embodiment, the linker is an electrode surface linker linked to the surface or electrode surface by irreversible or reversible immobilization. In another embodiment, the surface binding moiety (SBM) is immobilized to the surface or electrode surface by irreversible mobilization, wherein the irreversible mobilization is by covalent binding, cross-linking and/or entrapment. In a specific embodiment the surface binding moiety (SBM) is covalently bonded to the electrode surface. In another embodiment, the surface binding moiety (SBM) attaches by click chemistry, dithiol bond formation, Michael addition (for example, the use of an acrylamide linkage), a metal-sulfur bond linkage, nucleophilic substitution or enzyme catalyzed conjugation. In another embodiment, the immobilization or binding may be by physisorption.

In another embodiment, the surface binding moiety (SBM) may be or include a material binding protein or peptide. In a specific embodiment, the linker or electrode surface linker comprises a peptide for binding to the surface.

In another embodiment, the linker or electrode surface linker comprises a gold binding motif or binding peptide, a graphite/graphene binding motif or binding peptide, a PDMS binding motif or peptide, a silica binding motif or binding peptide, a cellulose binding motif or peptide, a polystyrene binding motif or peptide, a zinc oxide binding motif or binding peptide, a platinum binding motif or binding peptide, or a AuPd binding motif or binding peptide. In another embodiment, the linker or electrode surface linker comprises a copper binding motif or binding peptide, a palladium binding motif or binding peptide, a nickel binding motif or binding peptide, a zinc binding motif or binding peptide, an aluminum binding motif or binding peptide, a stainless steel binding motif or binding peptide, an iron oxide binding motif or binding peptide, a titanium oxide binding motif or binding peptide, a polypyrrol or a chlorine doped polypyrrole binding motif or binding peptide, or a polypropylene binding motif or binding peptide. In another embodiment, the linker or electrode surface linker comprises a titanium binding motif or binding peptide. In another embodiment, the linker or electrode surface linker comprises an aluminium oxide, and or tin oxide binding motif or binding peptide. In a specific embodiment, the surface binding moiety of the linkers may comprise one or more of the material binding peptide (MBP) amino acid sequences from Table 1.

TABLE 1
MBP Sequences

| | | | | Molecular | | |
|---|---|---|---|---|---|---|
| | | | Length | weight | | SEQ ID |
| ID | Target | Peptide Sequence | (aa) | (Dalton) | Reference | NO: |
| EMT014 | Gold | MHGKTQATSGTIQSMHG | 42 | 4303.752 | (KULP; | SEQ ID |
| | | KTQATSGTIQSMHGKTQ | | | SARIKAYA; | NO: 1 |
| | | ATSGTIQS | | | EVANS, | |
| | | | | | 2004) | |
| EMT015 | Gold | MHGKTQATSGTIQSMHG | 98 | 10018.0684 | (BROWN, | SEQ ID |
| | | KTQATSGTIQSMHGKTQ | | | 1997) | NO: 2 |
| | | ATSGTIQSMHGKTQATS | | | | |
| | | GTIQSMHGKTQATSGTIQ | | | | |
| | | SMHGKTQATSGTIQSMH | | | | |
| | | GKTQATSGTIQS | | | | |
| EMT016 | Gold | WAGAKRLVLRRE | 12 | 1454.7371 | (HNILOVA; | SEQ ID |
| | | | | | OREN; | NO: 3 |
| | | | | | SEKER; | |
| | | | | | WILSON | |
| | | | | | et al., | |
| | | | | | 2008) | |
| EMT017 | Gold | HFSSWETQQG | 10 | 1206.2336 | (TANAKA; | SEQ ID |
| | | | | | HIKIBA; | NO: 4 |
| EMT018 | Gold | WYEKWQKANW | 10 | 1438.6042 | YAMAS | SEQ ID |
| | | | | | HITA; | NO: 5 |
| | | | | | MUTO | |
| | | | | | et al., | |
| | | | | | 2017) | |
| EMT019 | Gold | VSGSSPDS | 8 | 734.716 | (HUANG; | SEQ ID |
| | | | | | CHIANG; | NO: 6 |
| | | | | | LEE; | |
| | | | | | GAO et | |
| | | | | | al., | |
| | | | | | 2005) | |
| EMT020 | Silicon | SSKKSGSYSGSKGSRRIL | 36 | 3541.8909 | (KRÖGER; | SEQ ID |
| | | GGGGMHGKTQATSGTIQ | | | DEUTZMANN; | NO: 7 |
| | | S | | | SUMPER, | |
| | | | | | 1999) | |
| EMT021 | Silicon | MSPHPHPRHHHTGGGGM | 30 | 3127.4165 | (NAIK; | SEQ ID |
| | | HGKTQATSGTIQS | | | BROTT; | NO: 8 |
| EMT022 | Silicon | RGRRRRLSCRLLGGGGM | 30 | 3198.6687 | CARSON; | SEQ ID |
| | | HGKTQATSGTIQS | | | AL., | NO: 9 |
| | | | | | 2012) | |
| EMT023 | Silicon | DSARGFKKPGKRGGGG | 30 | 3003.3374 | (COYLE; | SEQ ID |
| | | | | | BANEYX, | NO: 10 |
| | | MHGKTQATSGTIQS | | | 2016) | |
| EMT024 | Silicon | HPPMNASHPHMHGGGG | 30 | 3049.3582 | (ETESHOLA; | SEQ ID |
| | | MHGKTQATSGTIQS | | | BRILLSON; | NO: 11 |
| | | | | | LEE, | |
| | | | | | 2005) | |
| EMT025 | Silicon | HKDHHANQHVHMGGGG | 30 | 3147.4045 | (OKAMOTO; | SEQ ID |
| | | MHGKTQATSGTIQS | | | IWAHORI; | NO: 12 |
| | | | | | YAMAS | |
| | | | | | HITA, | |
| | | | | | 2019) | |
| EMT026 | Silicon | HPPMNASHPHMHGGGG | 16 | | | SEQ ID |
| | | | | | | NO: 13 |
| Cellulose | Cellulose | PTTGSCAVTYTANGWSG | 108 | | | SEQ ID |
| binding | | GFTAAVTLTNTGTTALSG | | | | NO: 14 |
| motif 1 | | WTLGFAFPSGQTLTQGW | | | | |
| | | SARWAQSGSSVTATNEA | | | | |
| | | WNAVLAPGASVEIGFSG | | | | |
| | | THTGTNTAPATFTVGGA | | | | |
| | | TCTTR | | | | |
| Cellulose | Cellulose | SGPAGCQVLWGVNQWN | 108 | | | SEQ ID |
| binding | | TGFTANVTVKNTSSAPV | | | | NO: 15 |
| motif 2 | | DGWTLTFSFPSGQQVTQ | | | | |
| | | AWSSTVTQSGSAVTVRN | | | | |
| | | APWNGSIPAGGTAQFGF | | | | |
| | | NGSHTGTNAAPTAFSLN | | | | |
| | | GTPCTVG | | | | |
| Polystyrene | Polystyrene | RAFIASRRIRRP | 12 | | | SEQ ID |
| binding | | | | | | NO: 16 |
| motif 1 | | | | | | |
| Polystyrene | Polystyrene | RIIIRRIR | 9 | | | SEQ ID |
| binding | | | | | | NO: 17 |
| motif 2 | | | | | | |
| Silica | Silica | RGRRRRLSCRLL | 12 | | | SEQ ID |
| Binding | | | | | | NO: 18 |
| Motif 1 | | | | | | |
| Silica | Silica | SSKKSGSYSGSKGSRRIL | 18 | | | SEQ ID |
| Binding | | | | | | NO: 19 |
| Motif 2 | | | | | | |
| Silica | Silica | MSPHPHPRHHHT | 12 | | | SEQ ID |
| Binding | | | | | | NO: 20 |
| Motif 3 | | | | | | |
| Silica | Silica | DSARGFKKPGKR | 12 | | | SEQ ID |
| Binding | | | | | | NO: 21 |
| Motif 4 | | | | | | |
| Silica | Silica | HPPMNASHPHMH | 12 | | | SEQ ID |
| Binding | | | | | | NO: 22 |
| Motif 5 | | | | | | |
| Silica | Silica | HKDHHANQHVHM | 12 | | | SEQ ID |
| Binding | | | | | | NO: 23 |
| Motif 6 | | | | | | |
| Silver | Silver | NPSSLFRYLPSD | 12 | | | SEQ ID |
| Binding | | | | | | NO: 24 |
| Motif 1 | | | | | | |
| Graphene | Graphene | HSSYWYAFNNKT | 12 | | | SEQ ID |
| Binding | | | | | | NO: 25 |
| Motif | | | | | | |
| Zinc | Zinc Oxide | EAHVMHKVAPRP | 12 | | | SEQ ID |
| Oxide | | | | | | NO: 26 |
| Binding | | | | | | |
| Motif 1 | | | | | | |
| Zinc | Zinc Oxide | HHGHSPTSPQVR | 12 | | | SEQ ID |
| Oxide | | | | | | NO: 27 |
| Binding | | | | | | |
| Motif 2 | | | | | | |
| PDMS | PDMS | MVMPGDNIKMVVTLIHPI | 30 | | | SEQ ID |
| Binding | (Polydimet | AMDDGLRFAIRE | | | | NO: 28 |
| Motif 1 | hylsiloxane) | | | | | |
| PDMS | PDMS | VGPNNVPYIVATITSNSA | 45 | | | SEQ ID |
| Binding | (Polydimet | GGQPVSLANLKAMYSIA | | | | NO: 29 |
| Motif 2 | hylsiloxane) | KKYDIPVVMD | | | | |
| PDMS | PDMS | NNSWTRVAFAGLKFQDV | 31 | | | SEQ ID |
| Binding | (Polydimet | GSFDYGRNYGVVYD | | | | NO: 30 |
| Motif 3 | hylsiloxane) | | | | | |
| Gold | Gold 1 | QVQLVESGAEVKKPGES | 120 | | | SEQ ID |
| binding | | LKISCKGSGYSFPSYWIN | | | | NO: 31 |
| motif 1 | | WVRQMPGKGLEWMGMI | | | | |
| | | YPADSDTRYSPSFQGHVT | | | | |
| | | ISADKSINTAYLQWAGLK | | | | |
| | | ASDTAIYYCARLGIGGRY | | | | |
| | | MSRWGQGTLVTVSSA | | | | |
| Platinum | Platinum | LEYKRGYKPR | 10 | | | SEQ ID |
| binding | | | | | | NO: 32 |
| motif 1 | | | | | | |
| AuPd | AuPd | DYKDDDDKAYSS | 20 | | | SEQ ID |
| binding | | GAPPMPPF | | | | NO: 33 |
| motif 1 | | | | | | |
| Ti binding | Titanium | RKLPDA | 6 | | | SEQ ID |
| motif 1 | | | | | | NO: 50 |
| Graphite/ | Graphite | EPLQLKM | 7 | | | SEQ ID |
| graphene | | | | | | NO: 54 |
| binding | | | | | | |
| peptide 1 | | | | | | |
| Graphite/ | Graphite | GAMHLPWHMGTL | 12 | | | SEQ ID |
| graphene | | | | | | NO: 55 |
| binding | | | | | | |
| peptide 2 | | | | | | |
| Graphite/ | Graphite | IMVTASSAYRRY | 12 | | | SEQ ID |
| graphene | | | | | | NO: 56 |
| binding | | | | | | |
| peptide 3 | | | | | | |
| Graphite/ | Graphite | VIAGASLWWSEKLVIA | 16 | | | SEQ ID |
| graphene | | | | | | NO: 57 |
| binding | | | | | | |
| peptide 4 | | | | | | |
| Graphite/ | Graphite | CALNNDEVDKFAM | 13 | | | SEQ ID |
| graphene | | | | | | NO: 58 |
| binding | | | | | | |
| peptide 5 | | | | | | |
| Graphite/ | Graphite | KKNYSSSISSIHC | 13 | | | SEQ ID |
| graphene | | | | | | NO: 59 |
| binding | | | | | | |
| peptide 6 | | | | | | |
| Graphite/ | Graphite | CGGHSSKLQFWYFWY | 15 | | | SEQ ID |
| graphene | | | | | | NO: 60 |
| binding | | | | | | |
| peptide 7 | | | | | | |
| Graphite/ | Graphite | HWSAWWIRSNQS | 12 | | | SEQ ID |
| graphene | | | | | | NO: 61 |
| binding | | | | | | |
| peptide 8 | | | | | | |
| Graphite/ | Graphite | IMVTESSDYSSY | 12 | | | SEQ ID |
| graphene | | | | | | NO: 62 |
| binding | | | | | | |
| peptide 9 | | | | | | |
| Graphite/ | Graphite | IMVTASSAYDDY | 12 | | | SEQ ID |
| graphene | | | | | | NO: 63 |
| binding | | | | | | |
| peptide 10 | | | | | | |
| Graphite/ | Graphite | IMVTQSSNYSSY | 12 | | | SEQ ID |
| graphene | | | | | | NO: 64 |
| binding | | | | | | |
| peptide 11 | | | | | | |
| Graphite/ | Graphite | IMVTKSSDYSSY | 12 | | | SEQ ID |
| graphene | | | | | | NO: 65 |
| binding | | | | | | |
| peptide 12 | | | | | | |
| Graphite/ | Graphite | HSSAAAAFNNKT | 12 | | | SEQ ID |
| graphene | | | | | | NO: 66 |
| binding | | | | | | |
| peptide 13 | | | | | | |
| Graphite/ | Graphite | HTSYWYAFNTKT | 12 | | | SEQ ID |
| graphene | | | | | | NO: 67 |
| binding | | | | | | |
| peptide 14 | | | | | | |
| Graphite/ | Graphite | YTTHVLPFAPSS | 12 | | | SEQ ID |
| graphene | | | | | | NO: 68 |
| binding | | | | | | |
| peptide 15 | | | | | | |
| Graphite/ | Graphite | HAWVDWIRPIH | 11 | | | SEQ ID |
| graphene | | | | | | NO: 69 |
| binding | | | | | | |
| peptide 16 | | | | | | |
| Graphite/ | Graphite | HWKHPWGAWDTL | 12 | | | SEQ ID |
| graphene | | | | | | NO: 70 |
| binding | | | | | | |
| peptide 17 | | | | | | |
| Graphite/ | Graphite | QQQLSTH | 7 | | | SEQ ID |
| graphene | | | | | | NO: 71 |
| binding | | | | | | |
| peptide 18 | | | | | | |
| Graphite/ | Graphite | GGPDSARGFKKPGKR | 18 | | | SEQ ID |
| graphene | | GPC | | | | NO: 72 |
| binding | | | | | | |
| peptide 19 | | | | | | |
| Cellulose | cellulose | GGGMHPNAGHGSLMR | 15 | | | SEQ ID |
| binding | | | | | | NO: 73 |
| peptide 1 | | | | | | |
| CBDCBH1 | cellulose | TPQSHYGQCGGGYSGPT | 36 | | | SEQ ID |
| | | VCASGTTCQVLNPYYSQ | | | | NO: 74 |
| | | CL | | | | |
| CBDClpC | | GVVSVQFNNGSSPASSNS | 157 | | | SEQ ID |
| | | IYARFKVTNTSGSPINLA | | | | NO: 75 |
| | | DLKLRYYYTQDADKPLT | | | | |
| | | FWCDHAGYMSGSNYID | | | | |
| | | ATSKVTGSFKAVSPAVT | | | | |
| | | NADHYLEVALNSDAGSL | | | | |
| | | PAGGSIEIQTRFARNDWS | | | | |
| | | NFDQSNDWSYTAAGSY | | | | |
| | | MDWQKISAFVGGTLAYG | | | | |
| | | STP | | | | |
| Ti binding | Titanium | QPYLFATDSLIK | 12 | | | SEQ ID |
| motif 2 | | | | | | NO: 76 |
| Ti binding | Titanium | GHTHYHAVRTQT | 12 | | | SEQ ID |
| motif 3 | | | | | | NO: 77 |
| Polystyrene | Polystyrene | RLLLRRLRR | 9 | | | SEQ ID |
| binding | | | | | | NO: 78 |
| motif 3 | | | | | | |
| Polystyrene | Polystyrene | KRAFIASRRIRRP | 13 | | | SEQ ID |
| binding | | | | | | NO: 79 |
| motif 4 | | | | | | |
| GSH | Copper | ECG | 3 | | (Slocik; | SEQ ID |
| | | | | | Wright, | NO: 80 |
| | | | | | 2003) | |
| HG12 | Copper | HGGGHGHGGGHG | 12 | | (Banerjee; | SEQ ID |
| | | | | | Yu; | NO: 81 |
| | | | | | Matsui, | |
| | | | | | 2003) | |
| Pd4 | Palladium | TSNAVHPTLRHL | 12 | | (Heinz; | SEQ ID |
| | | | | | Farmer; | NO: 82 |
| | | | | | Pandey; | |
| | | | | | Slocik et | |
| | | | | | al., | |
| | | | | | 2005) | |
| Pd2 | Palladium | NFMSLPRLGHMH | 12 | | (Heinz; | SEQ ID |
| | | | | | Farmer; | NO: 83 |
| | | | | | Pandey; | |
| | | | | | Slocik et | |
| | | | | | al., | |
| | | | | | 2005) | |
| HRE | Nickel | AHHAHHAAD | 9 | | (Slocik; | SEQ ID |
| | | | | | Wright, | NO: 84 |
| | | | | | 2003) | |
| SMN01 | Nickel | SGTGASY | 7 | | (Braun; | SEQ ID |
| | | | | | Bachmann; | NO: 85 |
| | | | | | Schonberger; | |
| | | | | | Matys et | |
| | | | | | al., | |
| | | | | | 2018) | |
| | Nickel | DAHKSEVA | 8 | | (Bar-Or; | SEQ ID |
| | | | | | Curtis; | NO: 86 |
| | | | | | Rao; | |
| | | | | | Bampos | |
| | | | | | et al., | |
| | | | | | 2001) | |
| A1-S1 | Aluminium | VPSSGPQDTRTT | 12 | | (Zuo; | SEQ ID |
| | | | | | Orken; | NO: 87 |
| | | | | | Wood, | |
| | | | | | 2005) | |
| A1-S2 | Aluminium | YSPDPRPWSSRY | 12 | | (Zuo; | SEQ ID |
| | | | | | Orken; | NO: 88 |
| | | | | | Wood, | |
| | | | | | 2005) | |
| | Zinc | SYHHHH | 6 | | (Mejare; | SEQ ID |
| | | | | | Bulow, | NO: 89 |
| | | | | | 2001) | |
| 46 | Zinc | ERSWTLDSALSM | 12 | | (Roth- | SEQ ID |
| | | | | | enstein; | NO: 90 |
| 83 | Zinc | SNNDLSPLQTSH | 12 | | Claasen; | SEQ ID |
| | | | | | Omiecienski; | NO: 91 |
| 21 | Zinc | DSSNPIFWRPSS | 12 | | Lammel | SEQ ID |
| | | | | | et al., | NO: 92 |
| 19 | Zinc | SILSTMSPHGAT | 12 | | 2012) | SEQ ID |
| | | | | | | NO: 93 |
| 26 | Zinc | SHALPLTWSTAA | 12 | | | SEQ ID |
| | | | | | | NO: 94 |
| 32 | Zinc | HVSIHRTTHHEM | 12 | | | SEQ ID |
| | | | | | | NO: 95 |
| 52 | Zinc | MKPDKAIRLDLL | 12 | | | SEQ ID |
| | | | | | | NO: 96 |
| 23 | Zinc | HYPTAKFHAERL | 12 | | | SEQ ID |
| | | | | | | NO: 97 |
| 20 | Zinc | TKNMLSLPVGPG | 12 | | | SEQ ID |
| | | | | | | NO: 98 |
| 22 | Zinc | FNTGSQMHQKFP | 12 | | | SEQ ID |
| | | | | | | NO: 99 |
| 53 | Zinc | HHTHR VDVHQTR | 12 | | | SEQ ID |
| | | | | | | NO: 100 |
| 29 | Zinc | FGLTAPRSASIL | 12 | | | SEQ ID |
| | | | | | | NO: 101 |
| 58 | Zinc | APRLPQSLLPQL | 12 | | | SEQ ID |
| | | | | | | NO: 102 |
| MS-S1 | Stainless | ATIHDAFYSAPE | 12 | | (Zuo; | SEQ ID |
| | | | | | Orken; | |
| | steel | | | | Wood, | NO: 103 |
| | | | | | 2005) | |
| MS-S2 | Stainless | NLNPNTASAMHV | 12 | | (Zuo; | SEQ ID |
| | | | | | Orken; | |
| | steel | | | | Wood, | NO: 104 |
| | | | | | 2005) | |
| MS-S3 | Stainless | NLTIASYPSMVV | 12 | | (Zuo; | SEQ ID |
| | | | | | Orken; | |
| | steel | | | | Wood, | NO: 105 |
| | | | | | 2005) | |
| MS-S4 | Stainless | QSHYRHISPAQV | 12 | | (Zuo; | SEQ ID |
| | steel | | | | Orken; | NO: 106 |
| | | | | | Wood, | |
| | | | | | 2005) | |
| MS-S5 | Stainless | QMDISLGRWSSM | 12 | | (Zuo; | SEQ ID |
| | steel | | | | Orken; | NO: 107 |
| | | | | | Wood, | |
| | | | | | 2005) | |
| MS-S6 | Stainless | YMKQIPAGRTNP | 12 | | (Zuo; | SEQ ID |
| | steel | | | | Orken; | NO: 108 |
| | | | | | Wood, | |
| | | | | | 2005) | |
| SBP-A | Stainless | VQHNTKYSVVIR | 12 | | (Mikami; | SEQ ID |
| | steel | | | | Fujimoto; | NO: 109 |
| | | | | | Taguchi; | |
| | | | | | Isao et | |
| | | | | | al., | |
| | | | | | 2020) | |
| SBP-B | Stainless | VQHNTKYSVVIR | 11 | | (Mikami; | SEQ ID |
| | steel | | | | Fujimoto; | NO: 111 |
| | | | | | Taguchi; | |
| | | | | | Isao et | |
| | | | | | al., | |
| | | | | | 2020) | |
| | Stainless | MTWDPSLASPRS | 12 | | (Vreuls; | SEQ ID |
| | steel | | | | Zocchi; | NO: 112 |
| | | | | | Genin; | |
| | | | | | Archambeau | |
| | | | | | et al., | |
| | | | | | 2010) | |
| | Stainless | YQLRPNAESLRF | 12 | | (Vreuls; | SEQ ID |
| | steel | | | | Zocchi; | NO: 113 |
| | | | | | Genin; | |
| | | | | | Archambeau | |
| | | | | | et al., | |
| | | | | | 2010) | |
| PAK(128- | Stainless | KCTSDQDEQFIPKGCSK | 17 | | (Cao; | SEQ ID |
| 144)ox | steel | | | | Fang; Yu; | NO: 114 |
| | | | | | Yong, | |
| | | | | | 2019) | |
| PilA from | Stainless | FTLIELMIVVAIIGILAAI | 150 | | (Giltner; | SEQ ID |
| Pseudomonas | steel | AIPAYQDYTARAQLSEAM | | | Shacik; | NO: 115 |
| aeruginosa | | TLASGLKTKVSDIFSQDG | | | Audette; | |
| (e.g., | | SCPANTAATAGIEKDTDI | | | Kao, et | |
| from | | NGKYVAKVTTGGTAAAS | | | al., | |
| K122-4) | | GGCTIVATMKASDVATP | | | 2005) | |
| | | LRGKTLTLTLGNADKGS | | | | |
| | | YTWACTSNADNKYLPKT | | | | |
| | | CQTATTTTP | | | | |
| K122-4 | Stainless | ACTSNADNKYLPKTCQT | 17 | | (Muruve; | SEQ ID |
| PilA | steel | | | | Cheng; | NO: 116 |
| receptor | | | | | Feng; | |
| binding | | | | | Liu et | |
| domain | | | | | al., | |
| from | | | | | 2016) | |
| Pseudomonas | | | | | | |
| aeruginosa | | | | | | |
| (e.g., | | | | | | |
| from | | | | | | |
| K122-4) | | | | | | |
| K122-4 | Stainless | Ac- | 17 | | (Davis; | SEQ ID |
| (128-144) | steel | ACTSNADNKYLPKTCQT- | | | Li; Irvin, | NO: 117 |
| Ox | | Amide | | | 2010) | |
| L-PAO | Stainless | Ac- | 17 | | (Davis; | SEQ ID |
| K130I | steel | ACISTQDPMFTPKGCDN- | | | Li; | NO: 118 |
| | | carboxyl | | | Shahrooei; | |
| | | | | | Yu, et | |
| | | | | | al., | |
| | | | | | 2013) | |
| L-PAO | Stainless | Ac- | 17 | | (Davis; | SEQ ID |
| | steel | ACKSTQDPMFTPKGCDN- | | | Li; | NO: 119 |
| | | carboxyl | | | Shahrooei; | |
| | | | | | Yu, et | |
| | | | | | al., | |
| | | | | | 2013) | |
| 1 | Iron Oxide | RRTVKHHVN | 9 | | (Sarikaya; | SEQ ID |
| | | | | | Tamerler; | NO: 120 |
| | | | | | Jen; | |
| | | | | | Schulten | |
| | | | | | et al., | |
| | | | | | 2003) | |
| | Iron Oxide | QMDTSTSLAPSR | 12 | | (Lower; | SEQ ID |
| | | | | | Lins; | NO: 121 |
| | | | | | Oestreicher; | |
| | | | | | Straatsma, | |
| | | | | | 2008) | |
| HRE | Titanium | AHHAHHAAD | 9 | | (Slocik; | SEQ ID |
| | Oxide | | | | Wright, | NO: 122 |
| | | | | | 2003) | |
| TiC1 | Titanium | CHKKPSKSC | 9 | | (Chen; | SEQ ID |
| | Oxide | | | | Su; | NO: 123 |
| | | | | | Neoh; | |
| | | | | | Choe, | |
| | | | | | 2006) | |
| SnBP01 | Aluminum | AGSWLRDIWTWLQSAL | 16 | | (Kim; | SEQ ID |
| | Oxide | | | | Jackman; | NO: 124 |
| | | | | | Mochizuki; | |
| | | | | | Yoon, et | |
| | | | | | al., | |
| | | | | | 2016) | |
| | Tin Oxide | LPPWKLK | 7 | | (Kilper; | SEQ ID |
| | | | | | Jahnke; | NO: 125 |
| | | | | | Wiegers; | |
| | | | | | Grohe, et | |
| | | | | | al., | |
| | | | | | 2019) | |
| φT59 | Chlorine | THRTSTLDYFVI | 12 | | (Sanghvi; | SEQ ID |
| | Doped | | | | Miller; | NO: 126 |
| | Polypyrrole | | | | Belcher; | |
| | | | | | Schmidt, | |
| | | | | | 2005) | |
| | Poly- | TSDIKSRSPHHR | 12 | | PATENT | SEQ ID |
| | propylene | | | | (Cunningham; | NO: 127 |
| | | | | | Lowe; | |
| | | | | | O'brien; | |
| | | | | | Wang et | |
| | | | | | al., | |
| | | | | | 2011) | |
| | Poly- | HTQNMRMYEPWF | 12 | | PATENT | SEQ ID |
| | propylene | | | | (Cunningham; | NO: 128 |
| | | | | | Lowe; | |
| | | | | | O'brien; | |
| | | | | | Wang et | |
| | | | | | al., | |
| | | | | | 2011) | |
| LCI | Poly- | AIKLVQSPNGNFAASFVL | 47 | | (Rubsam; | SEQ ID |
| | propylene | DGTKWIFKSKYYDSSKG | | | Weber; | NO: 129 |
| | | YWVGIYEVWDRK | | | Jakob; | |
| | | | | | Schwaneberg, | |
| | | | | | 2017) | |
| LCI-M1- | Poly- | AIKLVQSPNGNFAASFVL | 47 | | (Rubsam; | SEQ ID |
| PP | propylene | DGTKWTFKSKHYDSSKG | | | Weber; | NO: 130 |
| | | YWVGIYKVWDRK | | | Jakob; | |
| | | | | | Schwaneberg, | |
| | | | | | 2017) | |
| KR-2 | Poly- | AIKLVQSPNGNFAASFVL | 47 | | (Rubsam; | SEQ ID |
| | propylene | DGTKWIFKSKRYDSSKG | | | Davari; | NO: 131 |
| | | YWVGIYEVWRRK | | | Jakob; | |
| | | | | | Schwaneberg, | |
| | | | | | 2018) | |

In another embodiment, the present invention may comprise an amino acid sequence as in Table 1. In another embodiment, the present invention includes an amino acid sequence of about 75% to about 99.9% identical to one or more of SEQ ID NOs. 1-33, 50 or 54-131. In another embodiment, the present invention includes a nucleotide sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID NOs. 1-33, 50 or 54-131.

In another embodiment, electrode surface linker comprises a protein wherein the peptide or protein is a protein or peptide nanowire base.

The linkers may be conductive and may allow for electron transfer. In a specific embodiment, the linker that allows for electron transfer between the enzyme (such as an oxidoreductase enzyme) and the surface or electrode surface. In a specific embodiment the linkers are electrode surface linkers. In another specific embodiment, linker allows for electron transfer from the enzyme (such as an oxidoreductase enzyme) to the surface or electrode surface and/or for electron transfer from the surface to the enzyme.

In one embodiment, the linker may comprise a chemical nanowire, a means for chemical linkage, or a DNA nanowire type base. In a specific embodiment, the chemical nanowire base can include polymer containing compounds (e.g., MAL-PEG-NHS), cyclic carbon containing compounds (e.g., Succinimidyl 4-(N-maleimidomethyl)cyclohexane-1-carboxylate), or dithiol containing compounds (e.g., 1,6-hexanedithiol), or a means for a a metal-sulfur bond linkage.

In another specific embodiment, the linker may use, be, or include glutaraldehyde nanowires. In another specific embodiment, the nanowires may, use, be, or include SMCC Succinimidyl 4-(N-maleimidomethyl)cyclohexane-1-carboxylate, MBS (m-maleimidobenzoyl-N-hydroxysuccinimide ester) or a maleimide. In a specific embodiment, chemical modification may be used to link two portions of the linker or a portion of the linker to the surface, or a portion of the linker to the enzyme.

In another embodiment, the linker may comprise a DNA nanowire base, such as double stranded DNA, such as DNA composed of G and C bases ranging in length from about 4.08 nm to about 16 μm. In another embodiment, the linker may comprise a DNA nanowire base, such as double stranded DNA, such as DNA composed of G and C bases ranging in length from about 0.1 nm to about 10 μm or any length therebetween. In another embodiment, the length is from about 1.00 nm to about 1.0 μm. In another embodiment, the length is from about 1.00 nm to about 10 nm. In another embodiment, the length is from about 1.5 nm to about 5 nm. In another embodiment, the length is from about 1.00 nm to about 3 nm. In another embodiment, the length is about 1.00 nm, 2 nm, 3 nm, 4 nm, 5 nm, 6 nm, 7 nm, 8 nm, 9 nm, or 10 nm. In another embodiment, the DNA is single stranded DNA, such as single stranded DNA composed of G and C bases ranging in length from about 4.08 nm to about 16 μm. In another embodiment, the DNA is single stranded DNA, such as single stranded DNA composed of G and C bases ranging in length from about 0.1 nm to about 10 μm or any length therebetween. In another embodiment, the length is from about 1.00 nm to about 1.0 μm. In another embodiment, the length is from about 1.00 nm to about 10 nm. In another embodiment, the length is from about 1.5 nm to about 5 nm. In another embodiment, the length is from about 1.00 nm to about 3 nm. In another embodiment, the length is about 1.00 nm, 2 nm, 3 nm, 4 nm, 5 nm, 6 nm, 7 nm, 8 nm, 9 nm, or 10 nm.

In another embodiment, the DNA of the nanowire base is a polynucleotide of at least 2, 3, 4, 5, 6, 7, 8, 9, 10, 11, 12, 13, 14, or 15 nucleotides in length. In another embodiment, the polynucleotide sequences are single stranded or double stranded. In another embodiment, the nanowires comprise a nucleotide sequence of Table 3. In a specific embodiment, the nucleotide sequence may be any of the single stranded sequences of Table 3 or together as a double stranded sequence such as, for example, a double stranded sequence comprising SEQ ID NOs: 52 and 53.

TABLE 3
Nanowire Polynucleotide Sequences

| | Nucleotide Sequence | |
|---|---|---|
| ID | 5' to 3' | SEQ ID NO: |
| SSDNA1 | GGATGC | SEQ ID NO: 52 |
| SSDNA2 | GCATCC | SEQ ID NO: 53 |
| SSDNA3 | GCAACTAGGCTCG | SEQ ID NO: 132 |
| SSDNA4 | CGAGCCTAGTTGC | SEQ ID NO: 133 |
| SSDNA5 | GCACCTGAACCGCATGGACTCG | SEQ ID NO: 134 |
| SSDNA6 | CGAGTCCATGCGGTTCAGGTGC | SEQ ID NO: 135 |

In another embodiment, the present invention may comprise a nucleotide as in Table 3. In another embodiment, the present invention includes nucleotide sequence of about 75% to about 99.9% identical to one or more of SEQ ID Nos 52-53 or SEQ ID Nos 132-135. In another embodiment, the present invention includes a nucleotide sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID Nos 52-53 or SEQ ID Nos 132-135.

In another embodiment, the protein or peptide nanowire base includes a material binding peptide, a short conductive peptide, a metal-sulfur bond linkage, and/or one or more ferredoxins. In embodiments, the protein or peptide based nanowire comprises an iron-sulfur cluster containing conductive protein/peptide e.g., ferredoxin like structures/cytochrome like structures etc. In another embodiment, the one or more ferredoxins comprises a chain of ferredoxins. In another specific embodiment, the protein nanowire comprises ferredoxins, with 1 FeS cluster, more than 1 FeS cluster, a chain of ferrodoxins with 1 FeS clusters, a chain of ferrodoxins with more than 1 FeS clusters, or a chain of ferredoxins that includes a combination of ferredoxins with 1 FeS cluster and ferredoxins with >1 FeS cluster.

In another embodiment, the ferredoxin is from Chlamydomonas reinhardtii and/or Clostridium pasteurianum, in one or more clusters or chains as described above. In another embodiment, the linker or electrode surface linker may comprise one or more conductive proteins from E. coli. In a specific embodiment, the E. coli is E. coli C321, and the linker is the fimA protein. In another embodiment, the fimA protein is wildtype or a mutant. In a specific embodiment, the fimA is a mutant and point mutations at the following residues: A80, A109, and/or H82. In a specific embodiment, the mutations may be as follows: E. coli C321 fimA protein with A80F point mutation; E. coli C321 fimA protein with A109F point mutation; E. coli C321 fimA protein with A80F and A109F point mutation; E. coli C321 fimA protein with A80F, H82F and A109F point mutation; E. coli C321 fimA protein with A80F, H82F and A109Y point mutation; E. coli C321 fimA protein with A80Y point mutation; E. coli C321 fimA protein with A109Y point mutation; E. coli C321 fimA protein with A80Y and A109Y point mutations; E. coli C321 fimA protein with A80W point mutation; E. coli C321 fimA protein with A109W point mutation; E. coli C321 fimA protein with A80W and A109W point mutations; E. coli C321 fimA protein with A80Y, H82F and A80Y point mutations; E. coli C321 fimA protein with A80Y, H82Y and A109Y point mutations; or E. coli C321 fimA protein with a A109 2NaA point mutation.

In another embodiment, the linker or electrode surface linker may comprise one or more conductive proteins from pili/archaelum (e.g., from Syntrophus aciditrophicus, Methanospirillum hungatei, Geobacter metallireducens) or other conductive extracellular proteins (e.g., OmcS cytochrome, OmcZ cytochrome).

In another embodiment, the linkers comprise one or more short conductive peptides known in the art, including one or more conductive peptides with the amino acid sequence as in Table 2.

TABLE 2
Conductive Peptide Sequences

| ID | Peptide Sequence | SEQ ID NO: |
|---|---|---|
| Conductive | FTLIELLIVVAIIGILAAIAIPQFSAYRVKAYNSAA | SEQ ID NO: 34 |
| Peptide 1 | SSDLRNLKTALESAFADDQTYPPES | |
| Conductive | FTLIELMIVVAIIGILAAIAIPQYQNYVARSEGAS | SEQ ID NO: 35 |
| Peptide 2 | ALASVNPLKTTVEEALSRGWSVKSGTGTEDAT | |
| | KKEVPLGVAADANKLGTIALKPDPADGTADITL | |
| | TFTMGGAGPKNKGKIITLTRTAADGLWKCTSD | |
| | QDEQFIPKGCSR | |
| Conductive | MASNFKFKLLSQLKKRAEGGFTLIELLVVVIIIG | SEQ ID NO: 36 |
| Peptide 3 | VLAAIALPNLLGQVGKARESEAKSTIGALNRAQ | |
| | QGYFTEKGTFATDTETLEVPAPDGNFFSFAVNT | |
| | ADNTEAIQDATALNWEADGTRSMSGGTFYDSG | |
| | TRAFSTVVCRAEAGSEDTPPTPGGANDCGGAEV | |
| | IK | |
| Conductive | ELKAIAQEFKAIAKEFAIAFEFKAIAQK | SEQ ID NO: 37 |
| Peptide 4 | | |
| Conductive | YYACAYY | SEQ ID NO: 38 |
| Peptide 5 | | |
| Conductive | GNNQQNY | SEQ ID NO: 39 |
| Peptide 6 | | |
| Conductive | KVQIINKKL | SEQ ID NO: 40 |
| Peptide 7 | | |
| Conductive | VGGLG | SEQ ID NO: 41 |
| Peptide 8 | | |
| Conductive | VGGLGHHH | SEQ ID NO: 42 |
| Peptide 9 | | |
| Conductive | VGGLGWWW | SEQ ID NO: 43 |
| Peptide 10 | | |
| Conductive | VGGLGYYY | SEQ ID NO: 44 |
| Peptide 11 | | |
| Conductive | VGGLFFF | SEQ ID NO: 45 |
| Peptide 12 | | |
| Conductive | SVNVTQVGFP | SEQ ID NO: 46 |
| Peptide 13 | | |
| Conductive | SVNVTQVGFPHHH | SEQ ID NO: 47 |
| Peptide 14 | | |
| Conductive | SVNVTQVGFPWWW | SEQ ID NO: 48 |
| Peptide 15 | | |
| Conductive | SVNVTQVGFPYYY | SEQ ID NO: 49 |
| Peptide 16 | | |
| Conductive | PPPYPPPWC | SEQ ID NO: 51 |
| peptide 17 | | |
| Nanowire 1 | GS | SEQ ID NO: 136 |
| Nanowire 2 | VDGGGGGS | SEQ ID NO: 137 |
| Nanowire 3 | GTGGGGVDGGGGGS | SEQ ID NO: 138 |
| Nanowire 4 | GGGG | SEQ ID NO: 139 |
| Nanowire 5 | GGGGG | SEQ ID NO: 140 |
| Nanowire 6 | GGGGGG | SEQ ID NO: 141 |
| Nanowire 7 | GGGGGGGG | SEQ ID NO: 142 |
| Nanowire 8 | GGS | SEQ ID NO: 143 |
| Nanowire 9 | GGGS | SEQ ID NO: 144 |
| Nanowire 10 | GGGGS | SEQ ID NO: 145 |
| Nanowire 11 | PAPAP | SEQ ID NO: 146 |
| Nanowire 12 | EAAAK | SEQ ID NO: 147 |
| Nanowire 13 | GGYGG | SEQ ID NO: 148 |
| Nanowire 14 | GGFGG | SEQ ID NO: 149 |
| Nanowire 15 | GGFFGG | SEQ ID NO: 150 |
| Nanowire 16 | GGYYGG | SEQ ID NO: 151 |
| Nanowire 18 | GGSYWGGS | SEQ ID NO: 152 |
| Nanowire 19 | GGGGSEAAAKGGGS | SEQ ID NO: 153 |
| Nanowire 20 | GGGGSEAAAKEAAAKGGGS | SEQ ID NO: 154 |

In another embodiment, the present invention may comprise a peptide or an amino acid sequence as in Table 2. In another specific embodiment, the peptide consists of the amino acid sequences as in Table 2. In another embodiment, the present invention includes a peptide or an amino acid sequence of about 75% to about 99.9% identical to one or more of SEQ ID Nos 34-49 or 51. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID Nos 34-49 or 51.

In another embodiment, the present invention includes a peptide or an amino acid sequence of about 75% to about 99.9% identical to SEQ ID No. 49. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 49.

In another embodiment, the present invention includes a peptide or an amino acid sequence of about 75% to about 99.9% identical to SEQ ID No. 48. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 48.

In another embodiment, the present invention includes a peptide or an amino acid sequence of about 75% to about 99.9% identical to SEQ ID No. 47. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 47.

In another embodiment, the present invention includes a peptide or an amino acid sequence of about 75% to about 99.9% identical to SEQ ID No. 51. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to SEQ ID No. 51.

In another embodiment, the present invention includes a peptide or an amino acid sequence of about 75% to about 99.9% identical to one or more of SEQ ID Nos 136-154. In another embodiment, the present invention includes a peptide or an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID Nos 136-154.

In another specific embodiment, the present invention includes amino sequences or peptides that are repeats of SEQ ID Nos 136-154. For example, the peptide could be a repeat of any sequence of SEQ ID Nos 136-154 such as a repeat of 1-8 repeats, 1-6 repeats, 1-5, repeats, 1-4 repeats, or 1-2 repeats. In a specific embodiment, the peptides may be repeats such as (GS) n (n=1-8) (SEQ ID NO: 176); (GGS)ₙ (n=1-5) (SEQ ID NO: 177); (GGGS)ₙ (n=1-4) (SEQ ID NO: 178); (GGGGS)ₙ (n=1-4) (SEQ ID NO: 179); or (EAAAK)ₙ (n=1-4) (SEQ ID NO: 180).

The linkers and electrode surface linkers of the present invention may also include any appropriate amino acid sequence required to control steric hindrance and/or chemical interactions (organic or inorganic materials, peptides and proteins, cross-linking reagents, etc.).

The linker sequences of the present invention may include one or more passive linkers and/or active linkers. In certain embodiments, a passive linker is fused to an active linker, e.g., to link the SBM to the active portion of the linker. As used herein, a passive linker does not specifically bind to the surface or enzyme and is typically present between two polypeptide sequences to control steric hindrance, e.g., to retain activity of the two linked polypeptides. In particular embodiments, a passive linker may be a single bond or an amino acid sequence that links the SBM to another portion of the linker or directly to the enzyme. A passive linker may also be present between the enzyme and a member of a binding pair to which it is fused. The linker may be a covalent bond, an ionic bond, a non-covalent bond such as with the use of high-affinity molecules.

As used herein, an active linker may be fused to the SBM and/or enzyme and may be present to functionally link the SBM to the enzyme. In particular embodiments, an active linker binds to the enzyme. In certain embodiments, an active linker is a member of a binding pair, such as streptavidin/biotin. The link may be a covalent bond, an ionic bond, a non-covalent bond such as with the use of high-affinity molecules.

In another embodiment, the linker sequence may include other amino acid sequences, such as passive linkers, a linear tandem repeat polypeptides, a linear non-repeating polypeptides or linkers that allow for additional flexibility or rigidity to the overall linker system.

In one embodiment, the linker or electrode surface linker binds to the enzyme or an oxidoreductase enzyme. In a specific embodiment, the enzyme is an enzyme that produces hydrogen peroxide.

As described above the linker or electrode surface linker may comprise a surface binding moiety (SBM) that binds to the surface. The SBM may then also bind to a separate portion of the linker, e.g., a passive portion or active portion of the linker, or to the enzyme directly.

The linker or electrode surface linker thus provides a means for binding to the enzyme such as an enzyme that produces hydrogen peroxide, which may include various methods, including irreversible and reversible immobilization of the linker to the enzyme. Irreversible immobilization includes covalent binding, cross-linking and entrapment, while reversible methods include random physisorption, bioaffinity (biotin/streptavidin and protein A/G), a metal-sulfur bond linkage, chelation/metal binding and disulfide bonds (LIÉBANA; DRAGO, 2016).

In a specific embodiment, the linker is an electrode surface linker linked to the enzyme such as an enzyme that produces hydrogen peroxide, by irreversible or reversible immobilization. In another embodiment, linker, including potentially the SBM of the linker, is immobilized to the enzyme by irreversible mobilization, wherein the irreversible mobilization is by covalent binding, cross-linking and/or entrapment. In a specific embodiment the surface binding moiety (SBM) or another portion of the linker is covalently bonded to the enzyme, such as such as an enzyme that produces hydrogen peroxide. In another embodiment, the surface binding moiety (SBM) or another portion of the linker is covalently attached by click chemistry, dithiol bond formation, Michael addition (for example, the use of an acrylamide linkage), a metal-sulfur bond linkage, nucleophilic substitution or enzyme catalyzed conjugation.

The link to the enzyme may be a covalent bond, an ionic bond, a non-covalent bond such as with the use of high-affinity molecules.

In embodiments, the linker is covalently or non-covalently attached to the enzyme. For example, in embodiments, the linker is covalently attached to the enzyme by continuous protein expression, click chemistry, dithiol bond formation, Michael addition, nucleophilic substitution, a metal-sulfur bond linkage, a metal-nitrogen bond linkage, a metal-oxygen bond linkage, EDC coupling, or enzyme catalyzed conjugation. In embodiments, the linker is non-covalently attached to the enzyme by high affinity binding. In a specific embodiment, the enzyme is an enzyme that produces hydrogen peroxide. In a specific embodiment, the enzyme that produces hydrogen peroxide is methane monooxygenase reductase subunit, and/or NAD(P)H oxidase.

In a specific embodiment, the high affinity molecule in the linker may be an amino acid sequence comprising protein G from Streptococcus, or an amino acid sequence comprising streptavidin from Streptomyces for binding to the enzyme. In another embodiment, the linker comprises a binding tag, such as a His tag or biotin tag or other tag that allows for binding to the enzyme. In another embodiment, the tags may be on the enzyme, and therein the linker comprises the portion for binding to the tag. In other words, the linker may comprise streptavidin or biotin or the enzyme may comprise biotin or streptavidin respectively. In a specific embodiment, the enzyme is an enzyme that produces hydrogen peroxide. In a specific embodiment, the enzyme that produces hydrogen peroxide is methane monooxygenase reductase subunit, and/or NAD(P)H oxidase.

### Enzymes and Enzymatic Pathways of the Present Invention

Enzymatic bioelectrocatalysis is a specific form of electrocatalysis using an enzyme for catalyzing a certain reactions. In one general example of enzymatic bioelectrocatalysis, enzymes are associated with an electrode, including electrode linkers, in a manner that allows electron transfer between the electrode and the enzymes. Such electron transfer allows the continued function of each enzyme over many catalyzed reactions, including a series of reaction to obtain a final desired compound or product. The term “enzyme(s)” as used herein thus relates to a biologically based catalytic mechanism, and can comprise a protein that is both wild-type or mutated for any intended reaction by the user. Nonlimiting examples of other biologically based catalytic materials can include eukaryotic cells, prokaryotic cells, cellular organelles, nucleic acid enzymes (i.e. deoxyribozymes), and the like.

Oxidoreductase enzymes are biocatalytic proteins that can catalyse the coupled oxidation and reduction with a substrate, thus, transferring an electron(s) with the involvement of an electrode linker and/or a cofactor of the enzyme. In a specific embodiment of the present invention, the oxidoreductase enzyme can be used in a single reaction or in a series of reactions with other enzymes or oxidoreductase enzymes wherein the oxidoreductase enzyme is directly linked to an electrode linker. In a specific embodiment, the enzyme or oxidoreductase enzyme is directly linked to an electrode linker in a device, biodevice or reactor cell that contains the reaction or series of reactions if more than one enzyme or oxidoreductase enzyme is used.

In a specific embodiment, the enzyme can be an oxidoreductase enzyme. In another embodiment, the enzyme and/or oxidoreductase enzyme is one of a set or multiple enzymes and/or oxidoreductase enzymes. In another embodiment, the set of or multiple enzymes and/or oxidoreductase enzymes or apart of an enzymatic pathway.

In a specific embodiment, the enzyme may be an any enzyme that produces hydrogen peroxide, either as the primary reaction or product, or as a second or minor reaction or product. In a specific embodiment, the enzyme produces hydrogen peroxide from oxygen. In another embodiment, the enzyme that produces hydrogen peroxide may also produce another product, such as for example, glucose oxidase, alcohol oxidase, formate oxidase, sulfite oxidase, alditol oxidase, pyruvate oxidase, lactate oxidase, amine oxidase, glycolate oxidase or combinations thereof. In a specific embodiment, the parallel use of another enzyme may produce hydrogen peroxide from oxygen. In another embodiment, the enzyme that produces hydrogen peroxide may do so without another enzyme, including no other enzyme in parallel or in a sequential reaction. In a specific embodiment, the enzyme that produces hydrogen peroxide from oxygen is methane monooxygenase reductase subunit, and/or NAD(P)H oxidase.

In a specific embodiment, the enzyme that produces hydrogen peroxide may be a mutant enzyme. In a specific embodiment, the mutations in the enzyme may produce new functional groups near the surface of the enzyme, such as an amino acid mutated to a cysteine or a lysine. In another specific embodiment, the mutations may enhance the size, intended activity, or stability of the hydrogen peroxide producing enzyme.

In a specific embodiment, the enzyme is a methane monooxygenase reductase. In a specific embodiment, the methane monooxygenase reductase is from Methylomonas methanica. In another specific embodiment, the enzyme is a NAD(P)H Oxidase. In a specific embodiment, the NAD(P)H Oxidase is from Thermus thermophilus. In another specific embodiment, the enzyme that produces hydrogen peroxide may also produce superoxide, i.e., O₂⁻. In a specific embodiment, the superoxide forms into hydrogen peroxide.

In a specific embodiment, the enzyme is a NADH Oxidase. In a specific embodiment, the enzyme is Thermus thermophilus HB27 NADH Oxidase. In another specific embodiment, the enzyme is modified with a MBP and/or a nanowire or other form of linker. In another embodiment, the thermophilus HB27 strain is any strain known in the art. This may include for example variations at amino acid residue 194, 166, and/or 174 of Thermus thermophilus HB27 NADH Oxidase. In a specific embodiment, the variation can include as follows: K166, H174, and/or H194. In another specific embodiment, the NADH oxidase enzyme is stable at lower temperatures. In another specific embodiment, the enzyme may be a flavoprotein oxidoreductase. In another specific embodiment, the enzyme may be one or enzymes provided in Table 4.

TABLE 4
Enzymes and Enzyme Fusions

| Description | Amino Acid Sequence | SEQ ID NO: |
|---|---|---|
| Thermus thermophilus | MEATLPVLDAKTAALKRRSIRRYRKDPV | SEQ ID NO: 155 |
| HB27 NADH Oxidase | PEGLLREILEAALRAPSAWNLQPWRIVVV | |
| | RDPATKRALREAAFGQAHVEEAPVVLVL | |
| | YADLEDALAHLDEVIHPGVQGERREAQK | |
| | QAIQRAFAAMGQEARKAWASGQSYILLG | |
| | YLLLLLEAYGLGSVPMLGFDPERVKAILG | |
| | LPSHAAIPALVALGYPAEEGYPSHRLPLE | |
| | RVVLWR | |
| Thermus thermophilus | MNLNPNTASAMHVEATLPVLDAKTAAL | SEQ ID NO: 156 |
| HB27 NADH Oxidase | KRRSIRRYRKDPVPEGLLREILEAALRAPS | |
| with N terminal | AWNLQPWRIVVVRDPATKRALREAAFG | |
| stainless steel binding | QAHVEEAPVVLVLYADLEDALAHLDEVI | |
| peptide (SEQ ID NO. | HPGVQGERREAQKQAIQRAFAAMGQEA | |
| 104). | RKAWASGQSYILLGYLLLLLEAYGLGSV | |
| | PMLGFDPERVKAILGLPSHAAIPALVALG | |
| | YPAEEGYPSHRLPLERVVLWR | |
| Thermus thermophilus | MEATLPVLDAKTAALKRRSIRRYRKDPV | SEQ ID NO: 157 |
| HB27 NADH Oxidase | PEGLLREILEAALRAPSAWNLQPWRIVVV | |
| with C terminal | RDPATKRALREAAFGQAHVEEAPVVLVL | |
| stainless steel binding | YADLEDALAHLDEVIHPGVQGERREAQK | |
| peptide (SEQ ID NO. | QAIQRAFAAMGQEARKAWASGQSYILLG | |
| 104). | YLLLLLEAYGLGSVPMLGFDPERVKAILG | |
| | LPSHAAIPALVALGYPAEEGYPSHRLPLE | |
| | RVVLWRNLNPNTASAMHV | |
| Thermus thermophilus | MEATLPVLDAKTAALKRRSIRRYRKDPV | SEQ ID NO: 158 |
| HB27 NADH Oxidase | PEGLLREILEAALRAPSAWNLQPWRIVVV | |
| with C terminal | RDPATKRALREAAFGQAHVEEAPVVLVL | |
| peptide nanowire (GS) | YADLEDALAHLDEVIHPGVQGERREAQK | |
| and graphite binding | QAIQRAFAAMGQEARKAWASGQSYILLG | |
| peptide (SEQ ID NO. | YLLLLLEAYGLGSVPMLGFDPERVKAILG | |
| 104). | LPSHAAIPALVALGYPAEEGYPSHRLPLE | |
| | RVVLWRGSHSSYWYAFNNKT | |
| Thermus thermophilus | MEATLPVLDAKTAALKRRSIRRYRKDPV | SEQ ID NO: 159 |
| HB27 NADH Oxidase | PEGLLREILEAALRAPSAWNLQPWRIVVV | |
| (Y194 variation) | RDPATKRALREAAFGQAHVEEAPVVLVL | |
| | YADLEDALAHLDEVIHPGVQGERREAQK | |
| | QAIQRAFAAMGQEARKAWASGQSYILLG | |
| | YLLLLLEAYGLGSVPMLGFDPERVKAILG | |
| | LPSHAAIPALVALGYPAEEGYPSYRLPLE | |
| | RVVLWR | |
| Thermus thermophilus | MEATLPVLDAKTAALKRRSIRRYRKDPV | SEQ ID NO: 160 |
| HB27 NADH Oxidase | PEGLLREILEAALRAPSAWNLQPWRIVVV | |
| (K166/R174/Y194) | RDPATKRALREAAFGQAHVEEAPVVLVL | |
| | YADLEDALAHLDEVIHPGVQGERREAQK | |
| | QAIQRAFAAMGQEARKAWASGQSYILLG | |
| | YLLLLLEAYGLGSVPMLGFDPERVKAIL | |
| | GLPSRAAIPALVALGYPAEEGYPSYRLPL | |
| | ERVVLWR | |
| Thermus thermophilus | MEATLPVLDAKTAALKRRSIRRYRKDPV | SEQ ID NO: 161 |
| HB27 NADH Oxidase | PEGLLREILEAALRAPSAWNLQPWRIVVV | |
| (K166/H174/Y194) | RDPATKRALREAAFGQAHVEEAPVVLVL | |
| | YADLEDALAHLDEVIHPGVQGERREAQK | |
| | QAIQRAFAAMGQEARKAWASGQSYILLG | |
| | YLLLLLEAYGLGSVPMLGFDPERVKAIL | |
| | GLPSHAAIPALVALGYPAEEGYPSYRLPL | |
| | ERVVLWR | |
| Thermus thermophilus | MEATLPVLDAKTAALKRRSIRRYRKDPV | SEQ ID NO: 162 |
| HB27 NADH Oxidase | PEGLLREILEAALRAPSAWNLQPWRIVVV | |
| (R166/H174/H194) | RDPATKRALREAAFGQAHVEEAPVVLVL | |
| | YADLEDALAHLDEVIHPGVQGERREAQK | |
| | QAIQRAFAAMGQEARKAWASGQSYILLG | |
| | YLLLLLEAYGLGSVPMLGFDPERVRAILG | |
| | LPSHAAIPALVALGYPAEEGYPSHRLPLE | |
| | RVVLWR | |
| Thermus thermophilus | MEATLPVLDAKTAALKRRSIRRYRKDPV | SEQ ID NO: 163 |
| HB27 NADH Oxidase | PEGLLREILEAALRAPSAWNLQPWRIVVV | |
| One or more of | RDPATKRALREAAFGQAHVEEAPVVLVL | |
| K166R, H174R, and | YADLEDALAHLDEVIHPGVQGERREAQK | |
| H194Y | QAIQRAFAAMGQEARKAWASGQSYILLG | |
| | YLLLLLEAYGLGSVPMLGFDPERV(K/R) | |
| | AILGLPS(H/R)AAIPALVALGYPAEEGYPS | |
| | (H/Y)RLPLERVVLWR | |
| Thermus thermophilus | MEATLPVLDAKTAALKRRSIRRYRKDPV | SEQ ID NO: 164 |
| HB27 NADH Oxidase | PEGLLREILEAALRAPSAWNLQPWRIVVV | |
| One or more of | RDPATKRALREAAFGQAHVEEAPVVLVL | |
| K166R, K166H, | YADLEDALAHLDEVIHPGVQGERREAQK | |
| K166F, K166Y, | QAIQRAFAAMGQEARKAWASGQSYILLG | |
| K166W, H174R, | YLLLLLEAYGLGSVPMLGFDPERV(K/R/ | |
| H174K, H174F, | H/F/Y/W)AILGLPS(H/R/K/F/Y/W) | |
| H174Y, H174W, | AAIPALVALGYPAEEGYPS | |
| H194R, H194K, | (H/Y/R/K/F/W)RLPLERVVLWR | |
| H194Y, H194F, | | |
| H194W | | |
| Amphibacillus xylanus | MLDKDIKQQLEQYLALLENDIVIKVSVG | SEQ ID NO: 165 |
| nox1 gene | DDKVSKDTLELVNEIADMSSMISVEETTL | |
| | ERTPSFSINRKGEPDSGVVFAGIPLGHEFT | |
| | SLVLALLQVSGRAPKVEASVIDAIKKVEG | |
| | KHDFVTYVSLTCQNCPDVVQALNLMSV | |
| | VNPNITHTMVEGSAFKDEVDRLNILAVPS | |
| | VYLNGEFMASGRMTIEDILGHLGSGIDAS | |
| | ELNEKDPFDVLVIGGGPAGASSAIYAARK | |
| | GIRVGIIADRFGGQVMDTLGIENFIGTKYT | |
| | EGPKLISEIEQHVNEYNIDVMKGLRAHNI | |
| | EKKDLFEVQLDNGAVLKSKTIILATGAR | |
| | WRDIGVPGEKEYKNKGVAYCTHCDAPL | |
| | FEGKHVAVVGGGNSGIESALDLAGIVKH | |
| | VTVFEFMSELKADAVLQERLRSLPNVDVI | |
| | LNAQTTEITGDETVKGISYIDRTTNEEKH | |
| | VELQGAFIQIGLAPNTEWLGDTVERNQIG | |
| | EIVIDKKGQTSVPGIFAAGDCTDTPYKQII | |
| | VSMGAGATAALGAFDYLLRN | |
| Archaeoglobus | MKLFEPIEFGGMKVKNRIVMPAAELNYH | SEQ ID NO: 166 |
| fulgidus | TPDGRPTDRLLRFYEERAKGGIGFAVVGI | |
| AF_0455 gene | AKIEPHFFGGIAAHSDEFIPDLKKIADVFH | |
| | KYDVKCALQLWHPGRYEISFDPNRQPVA | |
| | PSPIPPPIFTKRTPKELTKEEILQIEEEFADA | |
| | AVRAKKAGFDAVELIGSAGYLISQFFSPA | |
| | TNKRTDEYGGSLENRTRFAVEIIQKIKEK | |
| | CGESYPVMIRIPGDEFIEGGNTVREMKQI | |
| | AKILEDAGVVAINVMAGWHESRKPLTT | |
| | MLVPRGGFAYLAAEIKSAVSVPVIASHRI | |
| | NDPIVAEKILQEGKADMVAMLRALIADP | |
| | ELPKKAKEGRFDEIRYCVACNQGCMDM | |
| | VMQAQPVTCLVNPIVGREAEFENLKAEK | |
| | PKKVVIVGGGPGGCMAAEMLARKGHKV | |
| | VLFEKTDKLGGQLNLAAKSPLGYEFAEV | |
| | GKYFMNVLPKLGVEVRYNTEADAGKVL | |
| | AENPDVAVIAVGASPLIPPIPGVENAVTAF | |
| | DVLAGRAEVGNSVVVIGGGGVGCDVAA | |
| | ELANEGKKVTIVEMLPKIGQDIGISTRWT | |
| | VLMYLKEKGVEMLTNTKAVEIKPNAVV | |
| | VEQNGERKELQCDTAVIAVGTKPNNGLY | |
| | DELQGKVSELYKIGDCVKPRKALDATRE | |
| | GADLALKV | |
| Bacillus subtilis | MNNTIETILNHRSIRSFTDQLLTAEEIDTL | SEQ ID NO: 167 |
| nfrA1 gene | VKSAQAASTSSYVQAYSIIGVSDPEKKRE | |
| | LSVLAGNQPYVEKNGHFFVFCADLYRHQ | |
| | QLAEEKGEHISELLENTEMFMVSLIDAAL | |
| | AAQNMSIAAESMGLGICYIGGIRNELDKV | |
| | TEVLQTPDHVLPLFGLAVGHPANLSGKK | |
| | PRLPKQAVYHENTYNVNTDDFRHTMNT | |
| | YDKTISDYYRERTNGKREETWSDQILNF | |
| | MKQKPRTYLNDYVKEKGFNKN | |
| Lactococcus lactis | MRAIIIGSGAAGLTTASTIRKYNKDMEIV | SEQ ID NO: 168 |
| AhpF gene | VITKEKEIAYSPCAIPYVIEGAIKSFDDIIM | |
| | HTPEDYKRERNIDILTETTVIDVDSKNNKI | |
| | KCVDKDGNEFEMNYDYLVLATGAEPFIP | |
| | PIEGKDLDGVFKVRTIEDGRAILKYIEENG | |
| | CKKVAVVGAGAIGLEMAYGLKCRGLDV | |
| | LVVEMAPQVLPRFLDPDMAEIVQKYLEK | |
| | EGIKVMLSKPLEKIVGKEKVEAVYVDGK | |
| | LYDVDMVIMATGVRPNIELAKKAGCKIG | |
| | KFAIEVNEKMQTSIPNIYAVGDCVEVIDFI | |
| | TGEKTLSPFGTAAVRQGKVAGKNIAGVE | |
| | AKFYPVLNSAVSKIGDLEIGGTGLTAFSA | |
| | NLKRIPIVIGRAKALTRARYYPGGKEIEIK | |
| | MIFNEDGKVVGCQIVGGERVAERIDAMSI | |
| | AIFKKVSAEELANMEFCYAPPVSMVHEP | |
| | LSLAAEDALKKLSNK | |
| Priestia megaterium | MNEAIRTIQDHRSIRQYTDEAVSDEHLDT | SEQ ID NO: 169 |
| (basonym: Bacillus | IIQSAQSAASSINGQQVTIISVQDKEKKKK | |
| megaterium) | LSELAGNQAWIDQAPLFLIFCADFNRAKI | |
| NfrA2 gene | AAELNDAPLGVTDGLESILVGATDAGISL | |
| | EAATVAAESLGLGTVPIGGIRRKPLEVIEL | |
| | LDLPEYVFPVSGLVVGHPSDHSAKKPRLP | |
| | QAAVHHRESYNHDLKSLIQDYDAEMAE | |
| | YMKKRTNGADDRNWSQTVSAIYKTIYYP | |
| | EVRAMLEKQGFKFE | |
| Pyrococcus furiosus | MRIVVIGSGTAGSNFALFMRKLDRKAEIT | SEQ ID NO: 170 |
| Nox-1 gene | VIGKEETMQYSPCALPHVISGVIEKPEDVI | |
| | VFPNEFYEKQRIKLLLNTEAKKIDRERKV | |
| | VVTDKGEIPYDKLVIATGSKAFVPPIKGV | |
| | ENEGVFTLKSLEDVRKIKEFIKKRNPKNA | |
| | VVIGAGLIGLEGAEAFAKLGMKVTVVEL | |
| | LEHLLPTMLDKDIAKIVEENMRKYGVDF | |
| | KFGVGVDEIIGDPVEKVKVGEEEIDADIV | |
| | LVATGVRANVELAKEAGLEVNRGIVVNE | |
| | YLQTSDPDIYAIGDCAEVIDAVTGKRTLS | |
| | QLGTSAVRMAKVAAENIAGRNVKFRPVF | |
| | NTAITEIFDLEIGAFGITEERAKKEEIEVVV | |
| | GKFRGSTKPEYYPGGKPIVVKLIFRKEDR | |
| | RLIGAQIVGGERVWGRIMTLSALAQKGA | |
| | TVEDVVYLETAYAPPISPTIDPITIAAEMA | |
| | MRKL | |
| Streptococcus mutans | MALDAEIKEQLGQYLQLLECEIVLQAQL | SEQ ID NO: 171 |
| JC2 | KDDANSQKVKEFLQEIVAMSPMISLDEK | |
| Nox-1 | ELPRTPSFRIAKKGQESGVEFAGLPLGHE | |
| | FYFVYLGSVTGFRASAKVETDIVKRIQAV | |
| | DEPMHFETYVSLTCHNCPDVVQAFNIMS | |
| | VVNPNISHTMVEGGMFKDEIEAKGIMSV | |
| | PTVYKDGTEFTSGRASIEQLLDLIAGPLKE | |
| | DAFDDKGVFDVLVIGGGPAGNSAAIYAA | |
| | RKGVKTGLLAETMGGQVMETVGIENMI | |
| | GTPYVEGPQLMAQVEEHTKSYSVDIMKA | |
| | PRAKSIQKTDLVEVELDNGAHLKAKTAV | |
| | LALGAKWRKINVPGEKEFFNKGVTYCPH | |
| | CDGPLFTDKKVAVIGGGNSGLEAAIDLA | |
| | GLASHVYILEFLPELKADKILQDRAEALD | |
| | NITILTNVATKEIIGNDHVEGLRYSDRTTN | |
| | EEYLLDLEGVFVQIGLVPSTDWLKDSGL | |
| | ALNEKGEIIVAKDGATNIPAIFAAGDCTD | |
| | SAYKQIIISMGSGATAALGAFDYLIRN | |
| Sus scrofa | MRVVVIGAGVIGLSTALCIHERYHSVLQP | SEQ ID NO: 172 |
| DAO gene | LDVKVYADRFTPFTTTDVAAGLWQPYTS | |
| | EPSNPQEANWNQQTFNYLLSHIGSPNAA | |
| | NMGLTPVSGYNLFREAVPDPYWKDMVL | |
| | GFRKLTPRELDMFPDYRYGWFNTSLILEG | |
| | RKYLQWLTERLTERGVKFFLRKVESFEE | |
| | VARGGADVIINCTGVWAGVLQPDPLLQP | |
| | GRGQIIKVDAPWLKNFIITHDLERGIYNSP | |
| | YIIPGLQAVTLGGTFQVGNWNEINNIQDH | |
| | NTIWEGCCRLEPTLKDAKIVGEYTGFRPV | |
| | RPQVRLEREQLRFGSSNTEVIHNYGHGG | |
| | YGLTIHWGCALEVAKLFGKVLEERNLLT | |
| | MPPSHL | |
| Thermococcus | MKIVVVGSGTAGSNFALFMRKLDRKAEI | SEQ ID NO: 173 |
| kodakarensis KOD1 | TVIGKEPTMQYSPCALPHVVSGTIEKPEDI | |
| tk0304 gene | IVFPNEFYEKQKINLMLNTEAKAIDRERK | |
| | VVVTDKGEVPYDKLVLAVGSKAFIPPIKG | |
| | VENEGVFTLKSLDDVRRIKAYIAERKPKK | |
| | AVVIGAGLIGLEGAEAFAKLGMEVLIVEL | |
| | MDRLMPTMLDKDTAKLVQAEMEKYGV | |
| | SFRFGVGVSEIIGSPVRAVKIGDEEVPADL | |
| | VLVATGVRANTDLAKQAGLEVNRGIVV | |
| | NEHLQTSDPEVYAIGDCAEVIDAVTGKR | |
| | TLSQLGTSAVRMAKVAAEHIAGKDVSFR | |
| | PVFNTAITELFGLEIGTFGITEERAKKEDIE | |
| | VAVGKFKGSTKPEYYPGGKPITVKLIFRK | |
| | SDRKLIGGQIVGGERVWGRIMTLSALAQ | |
| | KGATVEDVAYLETAYAPPISPTIDPITVAA | |
| | EMAQRKLR | |
| Thermococcus | MKYDVVVIGGSAGGLTAAISAKRFYPDK | SEQ ID NO: 174 |
| kodakarensis KOD1 | SVLVIKKEDVSMIPCGIPYIFGTLRSVEDD | |
| tk1481 gene | VLPTERFLKPLGIDVLVDEVTEINPKSKTL | |
| | LTKSGREIGWEKLVLATGSRPQIPDIPGVE | |
| | LEGVYTVSKDYHYLKELKKRVEDAEKV | |
| | VIIGGGFIALEVGDEIRKLGKDVTIVVRSR | |
| | LLRNSFDPEFSEMIENRLKEVGINVVYGH | |
| | VERLVGRERVEGVKLVEGGEIPADLVILS | |
| | TGYRPNVELAVKTGLKVTRYGIWTDEY | |
| | MRTSCPDIFAVGDCVEHRDFFTGKPFPLM | |
| | LASTATFEARIAGANLFKLQIVRENRRTIG | |
| | AYSTHVAGLTLAAAGLTEEAAKREGFEV | |
| | IVGRATGPDRHPAKFEDTSMVTVKLIFSR | |
| | DRGAILGAQLAGGKSVGEMINVLALAIQ | |
| | KRLTASELYTLQIATHPLLTASPVGYQIL | |
| | QAAEDALAKLRAGA | |
| Thermus thermophilus | MEATLPVLDAKTAALKRRSIRRYRKDPV | SEQ ID NO: 175 |
| HB8 | PEGLLREILEAALRAPSAWNLQPWRIVVV | |
| Nox | RDPATKRALREAAFGQAHVEEAPVVLVL | |
| | YADLEDALAHLDEVIHPGVQGERREAQK | |
| | QAIQRAFAAMGQEARKAWASGQSYILLG | |
| | YLLLLLEAYGLGSVPMLGFDPERVRAILG | |
| | LPSHAAIPALVALGYPAEEGYPSHRLPLE | |
| | RVVLWR | |
| Methanocaldococcus | MRAIIIGSGAAGLTTASTIRKYNKDMEIV | SEQ ID NO: 181 |
| jannaschii | VITKEKEIAYSPCAIPYVIEGAIKSFDDIIM | |
| Nox gene | HTPEDYKRERNIDILTETTVIDVDSKNNKI | |
| | KCVDKDGNEFEMNYDYLVLATGAEPFIP | |
| | PIEGKDLDGVFKVRTIEDGRAILKYIEENG | |
| | CKKVAVVGAGAIGLEMAYGLKCRGLDV | |
| | LVVEMAPQVLPRFLDPDMAEIVQKYLEK | |
| | EGIKVMLSKPLEKIVGKEKVEAVYVDGK | |
| | LYDVDMVIMATGVRPNIELAKKAGCKIG | |
| | KFAIEVNEKMQTSIPNIYAVGDCVEVIDFI | |
| | TGEKTLSPFGTAAVRQGKVAGKNIAGVE | |
| | AKFYPVLNSAVSKIGDLEIGGTGLTAFSA | |
| | NLKRIPIVIGRAKALTRARYYPGGKEIEIK | |
| | MIFNEDGKVVGCQIVGGERVAERIDAMSI | |
| | AIFKKVSAEELANMEFCYAPPVSMVHEP | |
| | LSLAAEDALKKLSNK | |

In another embodiment, the present invention includes an enzyme with an amino acid sequence of about 75% to about 99.9% identical to one or more of SEQ ID Nos 155-175 and 181. In another embodiment, the present invention includes an enzyme with an amino acid sequence of about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 98%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, or about 99.9% to one or more of SEQ ID Nos 155-175 and 181.

In a specific embodiment, the source of oxygen for producing hydrogen peroxide may be from one or multiple sources. In one embodiment, the oxygen source is oxygen gas, dissolved oxygen, from air or specifically, compressed air, or combinations thereof. In another specific embodiment, the oxygen is at the anode of the enzymatic reactor cell and is recirculated as a reagent at the cathode. In another embodiment, the enzyme reactor cell may be as in any one of Examples 1-8 disclosed herein.

In a specific embodiment, the enzymes as mentioned above may be wild-type. In another specific embodiment, the enzymes may a mutant. In another embodiment, the enzymes may have one or more amino acid residues mutated to a cysteine residue. In another specific embodiment, the mutation to a cysteine residue is on the outer surface of the folded enzyme, thereby allowing easy access to binding or reactive groups for linking to the thiol of the cysteine residue. In another embodiment, the wildtype or mutant enzyme or enzymes are provided to produce hydrogen peroxide.

Embodiments of the present invention also include methods for producing hydrogen peroxide by adding a starting agent or starting substrate to an enzymatic reactor cell described herein. In another embodiment, the enzyme of the enzymatic reactor cell comprises an enzyme that produces hydrogen peroxide. In another embodiment, the starting agent or stating substrate is selected from oxygen. In another embodiment, the methods include steps or methods as described in Examples 1˜4 described herein. In a specific embodiment, the oxygen is produced in a reactor cell described herein in a temperature range between about 15-35° C.; pH range of about 3.5-7.5; a current density of about: 0.005 to 200 mA/cm² with a cathodic more negative than 0.28 V vs SHE.

### EXAMPLES

### Example 1: Producing H₂O₂ from Oxygen, where the Oxygen Source is Oxygen Gas

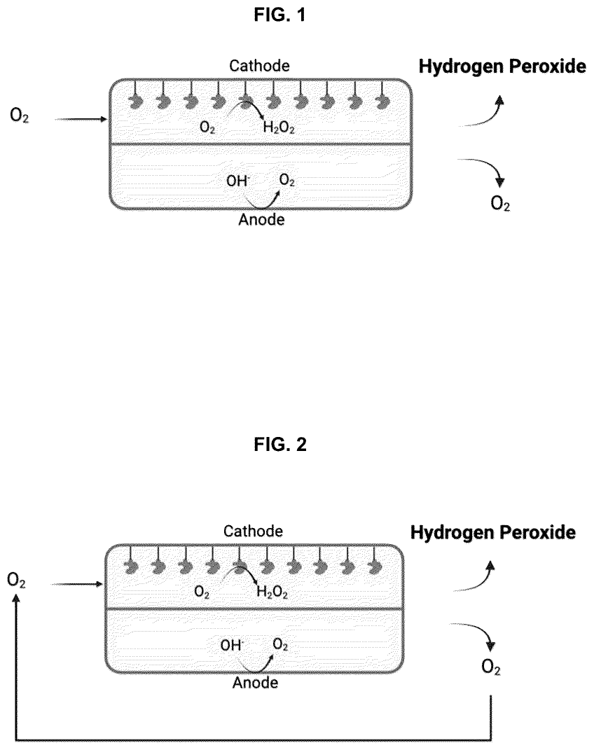

The following example utilizes a hydrogen peroxide producing enzyme to produce hydrogen peroxide in an enzymatic reactor cell (FIG. 1). In this example, the hydrogen peroxide producing enzyme is immobilized on the cathode surface by the use of a linker that specifically binds to the cathode surface by a surface binding moiety. Oxygen is added to the reactor by adding oxygen gas. The hydrogen peroxide producing enzyme uses the oxygen to produce hydrogen peroxide.

The produced hydrogen peroxide can be isolated and further concentrated by standard techniques such as vacuum distillation.

The hydrogen peroxide is produced in the reactor cells under the following conditions: temperature range 15-35° C.; pH range: 3.5-7.5; current density: 0.005 to 200 mA/cm² with a cathodic potential more negative than 0.28 vs SHE. The reactor cell provides a purity of 5-99 percent and a yield of 5-99 percent.

### Example 2: Producing H₂O₂ from Oxygen, where the Oxygen Source is Dissolved Oxygen

The following example utilizes a hydrogen peroxide producing enzyme to produce hydrogen peroxide in an enzymatic reactor cell (FIG. 1). In this example, the hydrogen peroxide producing enzyme is immobilized on the cathode surface by the use of a linker that specifically binds to the cathode surface by a surface binding moiety. Oxygen is added to the reactor by adding a dissolved oxygen containing electrolyte to the reactor. To do this, oxygen is added to the electrolyte prior to the electrolyte being added to the reactor. The hydrogen peroxide producing enzyme uses the oxygen to produce hydrogen peroxide.

The produced hydrogen peroxide can be isolated and further concentrated by standard techniques such as vacuum distillation.

The hydrogen peroxide is produced in the reactor cells under the following conditions: temperature range 15-35° C.; pH range: 3.5-7.5; current density: 0.005 to 200 mA/cm² with a cathodic potential more negative than 0.28 vs SHE. The reactor cell provides a purity of 5-99 percent and a yield of 5-99 percent.

### Example 3: Producing H₂O₂ from Oxygen, where the Oxygen Source is Air

The following example utilizes a hydrogen peroxide producing enzyme to produce hydrogen peroxide in an enzymatic reactor cell (FIG. 1). In this example, the hydrogen peroxide producing enzyme is immobilized on the cathode surface by the use of a linker that specifically binds to the cathode surface by a surface binding moiety. Oxygen is added to the reactor by adding air. The hydrogen peroxide producing enzyme uses the oxygen to produce hydrogen peroxide.

The produced hydrogen peroxide can be isolated and further concentrated by standard techniques such as vacuum distillation.

The hydrogen peroxide is produced in the reactor cells under the following conditions: temperature range 15-35° C.; pH range: 3.5-7.5; current density: 0.005 to 200 mA/cm² with a cathodic potential more negative than 0.28 vs SHE. The reactor cell provides a purity of 5-99 percent and a yield of 5-99 percent.

### Example 4: Producing H₂O₂ from Oxygen, where the Oxygen at the Anode is Recirculated to Act as a Reagent at the Cathode

The following example utilizes a hydrogen peroxide producing enzyme to produce hydrogen peroxide in an enzymatic reactor cell (FIG. 2). In this example, the hydrogen peroxide producing enzyme is immobilized on the cathode surface by the use of a linker that specifically binds to the cathode surface by a surface binding moiety. In this example, oxygen is produced at the anode.

In this example, oxygen is added to the cathodic side of the reactor by adding one, or a combination of, oxygen gas, electrolyte containing dissolved oxygen, air, and the oxygen produced at the anode. The hydrogen peroxide producing enzyme uses the oxygen to produce hydrogen peroxide.

In this example, oxygen is produced at the anode while hydrogen peroxide is produced at the cathode. This oxygen produced at the anode is recirculated to provide a source of oxygen at the cathode.

The produced hydrogen peroxide can be isolated and further concentrated by standard techniques such as vacuum distillation. The hydrogen peroxide is produced in the reactor cells under the following conditions: temperature range 15-35° C.; pH range: 3.5-7.5; current density: 0.005 to 200 mA/cm² with a cathodic potential more negative than 0.28 vs SHE. The reactor cell provides a purity of 5-99 percent and a yield of 5-99 percent.

### Example 5: Producing H₂O₂ when Enzyme and Linker are Absorbed to a Stainless Steel Sheet Surface

Enzyme production: The enzyme (Thermus thermophilus HB27 NADH Oxidase; fusion as SEQ ID NO. 156) was expressed recombinantly in E. coli using standard techniques. A plasmid was prepared encoding the enzyme gene and a material binding peptide (MBP) SEQ ID NO. 104 that binds to stainless steel, wherein the MBP and enzyme were expressed as a continuous construct. The MBP is at the N terminus and continuous construct was verified by sequencing. The verified plasmid was transformed into an expression strain of E. coli. Following initial growth of the E. coli, induction was performed and the cultures were permitted to grow for 2-24 hours for recombinant expression. Cells were isolated by centrifugation, lysed by sonication, and purified using a combination of heat incubation and column purification. Protein identity was verified with SDS-PAGE. Enzyme activity was confirmed with activity assays where the enzyme was mixed with NADH in 25 mM MOPS, 75 mM NaCl, pH 7.0. The change in absorbance over time was monitored at 340 nm with a plate reader.

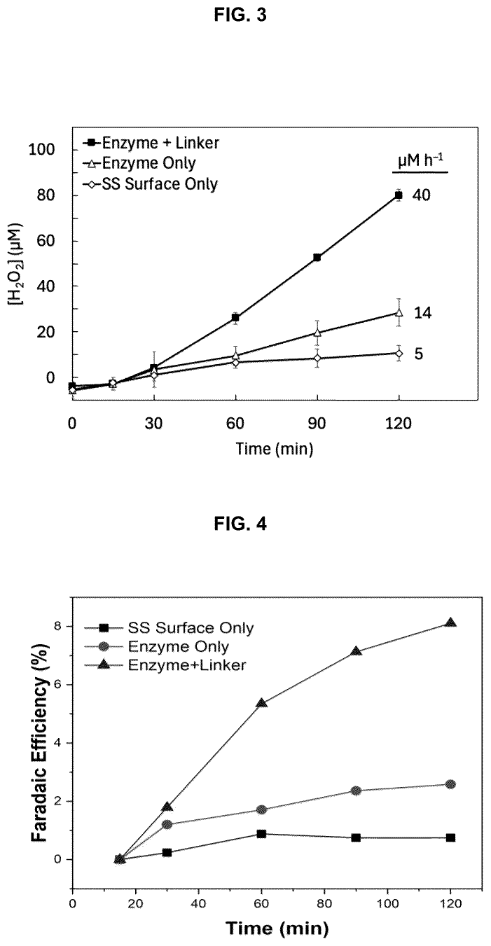

Bio-electrochemical hydrogen peroxide formation: Stainless steel (SS) sheets were cut into rectangular pieces measuring 1 cm×3 cm. The electrodes were cleaned by sequentially soaking them in isopropyl alcohol and Milli-Q water, followed by drying with argon (Ar) gas. The central region of the SS electrode was masked with Kapton tape, leaving two exposed areas: one measuring 0.5 cm×1 cm for electrochemical measurements and another measuring 0.5 cm×0.5 cm for electrical connections. Then the SS flag electrode was immersed in a three-electrode cell and subjected to a −3 mA current for 15 minutes to prepare the surface for deposition. The electrochemically pretreated SS electrode was placed in a small weigh boat, and 100 μM Enzyme+Linker, i.e., with MBP (supplemented with 1 mM Flavin) was deposited onto the exposed electrode surface. The electrode was incubated for 20 minutes, followed by rinsing in the electrolyte to remove excess enzyme. This electrode functioned as the working electrode. An undivided electrochemical cell was assembled using a platinum counter electrode and an Ag/AgCl reference electrode. The composition of the electrolyte was 25 mM MOPS and 75 mM NaCl at pH 7. The electrolyte was continuously stirred using a magnetic stirrer placed underneath the cell during electrochemical characterization. Chronoamperometry was performed at −0.8 V (vs. NHE) for 2 hours. Control experiments were conducted using the SS surface only and enzyme only for comparison (FIGS. 3 and 4). Specifically, FIG. 3 shows the production of H₂O₂ in μM over time when Enzyme+Linker, i.e., with MBP, is adsorbed to the SS disk surface in comparison to enzyme only or the SS surface only.

FIG. 4 specifically shows the faradaic efficiency expressed as a percentage over time for the production of H₂O₂ when Enzyme+Linker is adsorbed to the SS disk surface in comparison to enzyme only and SS surface only.

Product assay: Samples collected from electrochemical experiments and H₂O₂ standards prepared in the same matrix were combined with TiOSO₄ and H₂SO₄ in triplicate and allowed to react under ambient conditions. The absorbance at 405 nm was collected with a plate reader, calibration curve constructed from the standards, and the amount of H₂O₂ in the samples and their associated error determined using the calibration curve

### Example 6: Producing H₂O₂ when Enzyme and Linker are Absorbed to a Stainless Steel Sheet Surface

Enzyme production: The enzyme (Thermus thermophilus HB27 NADH Oxidase; fusion as SEQ ID NO: 157) was expressed recombinantly in E. coli using standard techniques. A plasmid was prepared encoding the enzyme gene and a material binding peptide (MBP) SEQ ID NO. 104 that binds to stainless steel, wherein the MBP and enzyme were expressed as a continuous construct. The MBP is at the C terminus and continuous construct was verified by sequencing. The verified plasmid was transformed into an expression strain of E. coli. Following initial growth of the E. coli, induction was performed and the cultures were permitted to grow for 2-24 hours for recombinant expression. Cells were isolated by centrifugation, lysed by sonication, and purified using a combination of heat incubation and column purification. Protein identity was verified with SDS-PAGE. Enzyme activity was confirmed with activity assays where the enzyme was mixed with NADH in 25 mM MOPS, 75 mM NaCl, pH 7.0. The change in absorbance over time was monitored at 340 nm with a plate reader.

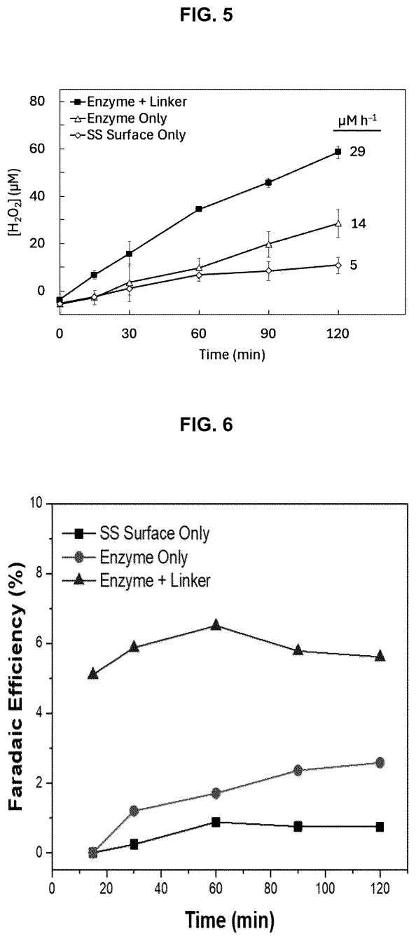

Bio-electrochemical hydrogen peroxide formation: Stainless steel (SS) sheets were cut into rectangular pieces measuring 1 cm×3 cm. The electrodes were cleaned by sequentially soaking them in isopropyl alcohol and Milli-Q water, followed by drying with argon (Ar) gas. The central region of the SS electrode was masked with Kapton tape, leaving two exposed areas: one measuring 0.5 cm×1 cm for electrochemical measurements and another measuring 0.5 cm×0.5 cm for electrical connections. Then the SS flag electrode was immersed in a three-electrode cell and subjected to a −3 mA current for 15 minutes to prepare the surface for deposition. The electrochemically pretreated SS electrode was placed in a small weigh boat, and 100 μM Enzyme+Linker, i.e., with MBP (supplemented with 1 mM Flavin) was deposited onto the exposed electrode surface. The electrode was incubated for 20 minutes, followed by rinsing in the electrolyte to remove excess enzyme. This electrode functioned as the working electrode. An undivided electrochemical cell was assembled using a platinum counter electrode and an Ag/AgCl reference electrode. The composition of the electrolyte was 25 mM MOPS and 75 mM NaCl at pH 7. The electrolyte was continuously stirred using a magnetic stirrer placed underneath the cell during electrochemical characterization. Chronoamperometry was performed at −0.8 V (vs. NHE) for 2 hours. Control experiments were conducted using the SS surface only and enzyme only for comparison (FIGS. 5 and 6). Specifically, FIG. 5 shows the production of H₂O₂ in μM over time when Enzyme+Linker is adsorbed to the SS disk surface in comparison to enzyme only or SS surface only. FIG. 6 shows the faradaic efficiency expressed as a percentage over time for the production of H₂O₂ when Enzyme+Linker, i.e., with MBP is adsorbed to the SS disk surface in comparison to enzyme only and SS surface only.

Product assay: Samples collected from electrochemical experiments and H₂O₂ standards prepared in the same matrix were combined with TiOSO₄ and H₂SO₄ in triplicate and allowed to react under ambient conditions. The absorbance at 405 nm was collected with a plate reader, calibration curve constructed from the standards, and the amount of H₂O₂ in the samples and their associated error determined using the calibration curve.

### Example 7: Producing H₂O₂ when Enzyme and Linker are Absorbed to a Glassy Carbon Disk Surface

Enzyme production: The enzyme (Thermus thermophilus HB27 NADH Oxidase; fusion as SEQ ID NO. 158) was expressed recombinantly in E. coli using standard techniques. A plasmid was prepared encoding the enzyme gene, amino acid linker sequence (nanowire GS amino acid sequence SEQ ID NO. 136), and material binding peptide (MBP) SEQ ID No. 25. The material binding peptide, nanowire, and enzyme are expressed as a continuous construct. The material binding peptide and nanowire are at the C terminus and was verified by sequencing. The verified plasmid was transformed into an expression strain of E. coli. Following initial growth of the E. coli, induction was performed and the cultures were permitted to grow for 2-24 hours for recombinant expression. Cells were isolated by centrifugation, lysed by sonication, and purified using a combination of heat incubation and column purification. Protein identity was verified with SDS-PAGE. Enzyme activity was confirmed with activity assays where the enzyme was mixed with NADH in 25 mM MOPS, 75 mM NaCl, pH 7.0. The change in absorbance over time was monitored at 340 nm with a plate reader.

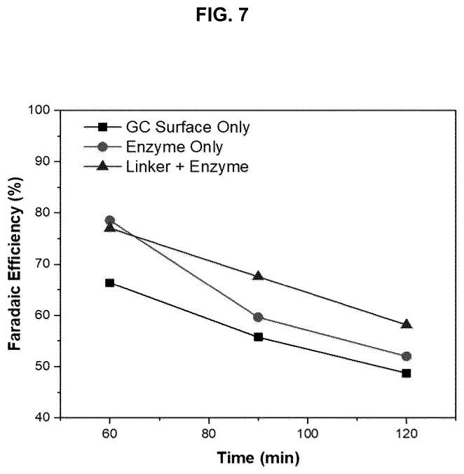

Bio-electrochemical hydrogen peroxide formation: A glassy carbon (GC) disk electrode (5 mm diameter) was cleaned and polished using 0.3 μm alumina powder, followed by sonication in isopropyl alcohol and Milli-Q water. The electrode was then dried under an argon (Ar) flow. The cleaned GC electrode was placed in a holder, and a volume of enzyme+linker sufficient to cover the electrode was deposited onto the surface (e.g., 5-20 μL). The electrode was incubated for 10-60 minutes and rinsed in the electrolyte to remove excess enzyme. The immobilized GC electrode was mounted on a rotating disk electrode (RDE) shaft, serving as the working electrode. An undivided RDE electrochemical cell was assembled using a platinum counter electrode and an Ag/AgCl reference electrode. The electrolyte consisted of 25 mM MOPS and 75 mM NaCl at pH 7. The electrolyte was continuously bubbled with oxygen during electrochemical characterization. Chronoamperometry was performed at −0.4 V (vs. NHE) for 2 hours. Control experiments were conducted using the GC surface only and enzyme alone for comparison (FIG. 7). Specifically, FIG. 7 shows the faradaic efficiency expressed as a percentage over time for the production of H₂O₂ when Enzyme+Linker is adsorbed to the GC disk surface in comparison to enzyme only and GC surface only.

Product assay: Samples collected from electrochemical experiments and H₂O₂ standards prepared in the same matrix were combined with TiOSO₄ and H₂SO₄ in triplicate and allowed to react under ambient conditions. The absorbance at 405 nm was collected with a plate reader, calibration curve constructed from the standards, and the amount of H₂O₂ in the samples and their associated error determined using the calibration curve.

### Example 8: Producing H₂O₂ when Enzyme and Linker are Absorbed to a Carbon Paper Surface

Enzyme production: The enzyme (Thermus thermophilus HB27 NADH Oxidase; fusion as SEQ ID NO. 158) was expressed recombinantly in E. coli using standard techniques. A plasmid was prepared encoding the enzyme gene, amino acid linker sequence (nanowire GS amino acid sequence; SEQ ID NO. 136), and material binding peptide (MBP) SEQ ID No. 25. The material binding peptide, nanowire, and enzyme are expressed as a continuous construct. The material binding peptide and nanowire are at the C terminus and was verified by sequencing. The verified plasmid was transformed into an expression strain of E. coli. Following initial growth of the E. coli, induction was performed and the cultures were permitted to grow for 2-24 hours for recombinant expression. Cells were isolated by centrifugation, lysed by sonication, and purified using a combination of heat incubation and column purification. Protein identity was verified with SDS-PAGE. Enzyme activity was confirmed with activity assays where the enzyme was mixed with NADH in 25 mM MOPS, 75 mM NaCl, pH 7.0. The change in absorbance over time was monitored at 340 nm with a plate reader.

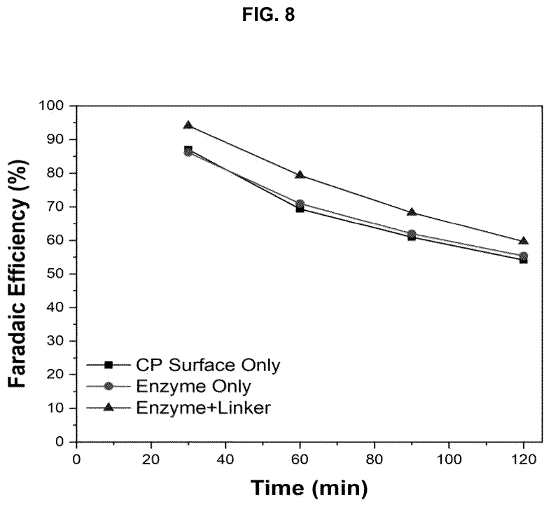

Bio-electrochemical hydrogen peroxide formation: Carbon paper (CP) was cut into thin strips measuring 0.5 cm×3 cm. The entire strip was coated with melted wax, leaving an uncoated 0.5 cm×0.5 cm area. The exposed CP surface was submerged in enzyme+linker solution and left in a refrigerator overnight for deposition. The electrode was then rinsed in the electrolyte for 10-60 minutes to remove excess enzyme. This conjugated enzyme-loaded electrode functioned as the working electrode. An undivided electrochemical cell was assembled using a platinum counter electrode and an Ag/AgCl reference electrode. The electrolyte consisted of 25 mM MOPS and 75 mM NaCl at pH 7. The electrolyte was continuously bubbled with oxygen during electrochemical characterization. Chronoamperometry was performed at −0.4 V (vs. NHE) for 2 hours. Experiments were conducted using the CP surface only and enzyme alone for comparison (FIG. 8). Specifically, FIG. 8 shows the faradaic efficiency expressed as a percentage over time for the production of H₂O₂ when Enzyme+Linker is adsorbed to the CP surface in comparison to enzyme only and CP surface only.

Product assay: Samples collected from electrochemical experiments and H₂O₂ standards prepared in the same matrix were combined with TiOSO₄ and H₂SO₄ in triplicate and allowed to react under ambient conditions. The absorbance at 405 nm was collected with a plate reader, calibration curve constructed from the standards, and the amount of H₂O₂ in the samples and their associated error determined using the calibration curve.

While preferred embodiments have been described above and illustrated in the accompanying drawings, it will be evident to those skilled in the art that modifications may be made without departing from this disclosure. Such modifications are considered as possible variants comprised in the scope of the disclosure.

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

1. An enzymatic reactor cell, comprising:
a surface, a linker, and one or more enzymes, wherein the surface is directly linked to the linker and the linker is further directly linked to the enzyme, wherein the enzymatic reactor cell produces superoxide and/or hydrogen peroxide.

2. The enzymatic reactor cell of claim 1, wherein the one or more enzymes comprises an enzyme that produces hydrogen peroxide and/or superoxide.

3. The enzymatic reactor cell of claim 2, wherein the enzyme that produces hydrogen peroxide and/or superoxide is a mutant form.

4. The enzymatic reactor cell of claim 2, wherein the enzyme that produces hydrogen peroxide and/or superoxide may also produce another product, such as the enzyme glucose oxidase, alcohol oxidase, formate oxidase, sulfite oxidase, alditol oxidase, pyruvate oxidase, lactate oxidase, amine oxidase, glycolate oxidase or combinations thereof.

5. The enzymatic reactor cell of claim 2, wherein the enzyme that produces hydrogen peroxide and/or superoxide is methane monooxygenase reductase subunit, a flavoprotein oxidoreductase, an NADH Oxidase and/or NAD(P)H oxidase.

6. The enzymatic reactor cell of claim 5, wherein the enzyme is an NADH Oxidase and/or a flavoprotein oxidoreductase from Table 4.

7. The enzymatic reactor cell of claim 1, wherein the enzymatic reactor cell further comprises a starting agent or starting substrate that is oxygen, and the oxygen source is oxygen gas, dissolved oxygen, from air or specifically, compressed air, or combinations thereof.

8. The enzymatic reactor cell of claim 1, wherein the surface is an electrode.

9. The enzymatic reactor cell of claim 1, wherein the surface is carbon based, an inorganic surface, a metal, a metal oxide, silica or silicon, comprises a polymer such as cellulose, polystyrene, PDMS, chlorine doped polypyrrole, polypropylene, is an alloy and/or is a combination thereof.

10. The enzymatic reactor cell of claim 9, wherein the surface is carbon based.

11. The enzymatic reactor cell of claim 10, wherein the carbon based surface comprises one or more from the following: graphite; graphene; Glassy carbon; Carbon/graphite felt; carbon/graphite paper; teflonated carbon paper; teflonated carbon felt; carbon based gas diffusion electrode containing a macroporous and/or microporous layer; SWCNT; MWCNT; activated carbon; graphite foil; Carbon nanofibers; Carbon nanotubes; Carbon black-coated graphite felt; Microporous carbon; Hierarchically porous carbon; and/or Mesoporous carbon; heteroatom doped carbon (e.g., oxygen/nitrogen/boron/sulphur/fluorine/—COOH/C—O—C doped carbon, oxidized graphite felt, oxygenated carbon nanotubes, quinone incorporated carbon nanostructures, edge oxygenated graphitic nanoplatelets, or nitrogen-rich layered graphene.

12. The enzymatic reactor cell of claim 1, wherein the linker comprises a peptide, protein, a chemical polymer, and/or polynucleotide.

13. The enzymatic reactor cell of claim 1, wherein the linker comprises a surface binding moiety (SBM).

14. The enzymatic reactor cell of claim 13, wherein the surface binding moiety (SBM) binds to or is immobilized to the surface, another portion of the linker, and/or the enzyme by non-covalent bonding, covalent bonding, physisorption, and/or high affinity binding.

15. The enzymatic reactor cell of claim 13, wherein the surface binding moiety (SBM) is covalently bonded to the surface, another portion of the linker, and/or the enzyme.

16. The enzymatic reactor cell of claim 15, wherein the surface binding moiety (SBM) is covalently bonded to the surface, another portion of the linker, and/or the enzyme by click chemistry, dithiol bond formation, Michael addition, nucleophilic substitution, a metal-sulfur bond linkage, a metal-nitrogen bond linkage, a metal-oxygen bond linkage, enzyme catalyzed conjugation, and/or EDC coupling.

17. The enzymatic reactor cell of claim 15, wherein the surface binding moiety (SBM) is covalently bonded by continuous protein expression wherein the surface binding moiety (SBM) is a continuous protein with another portion of the linker; the surface binding moiety (SBM) is a continuous protein with the enzyme; or the surface binding moiety (SBM) is a continuous protein with another portion of the linker, and/or the enzyme.

18. The enzymatic reactor cell of claim 1, wherein the linker binds to another portion of the linker, the enzyme, and/or the surface.

19. The enzymatic reactor cell of claim 18, wherein the linker is covalently bonded to another portion of the linker, the enzyme, and/or the surface by click chemistry, dithiol bond formation, Michael addition, nucleophilic substitution, a metal-sulfur bond linkage, a metal-nitrogen bond linkage, a metal-oxygen bond linkage, enzyme catalyzed conjugation, and/or EDC coupling.

20. The enzymatic reactor cell of any claim 13, wherein the SBM comprises a material binding peptide (MBP).

21. The enzymatic reactor cell of claim 20, wherein the material binding peptide (MBP) or surface binding moiety (SBM) of the linker comprises an amino acid sequence from Table 1.

22. The enzymatic reactor cell of claim 20, wherein the material binding peptide (MBP) binds to carbon base surfaces, metal, metal oxide, a polymer and/or an inorganic surface.

23. The enzymatic reactor cell of claim 20, wherein the MBP comprises SEQ ID NO. 25.

24. The enzymatic reactor cell of claim 1, wherein the linker comprises a peptide, protein, a chemical polymer, nanowire, polynucleotide or chemical means such as chemical nanowires to link a) portions of the linker together, b) the linker to the surface and/or c) the linker to the enzyme.

25. The enzymatic reactor cell of claim 24, wherein the chemical means or chemical nanowire may use for linking, or include a maleimide functional group, a tetrafluorophenyl functional group, an aldehyde functional group, an amine functional group, a N-hydroxysuccinimide functional group, a thiol functional group, a haloacetyl functional group, a pyridyl disulfide functional group, an imidoester functional group, an epoxide functional group, a hydrocarbon chain, one of more polyethylene glycol units, and/or one or more aromatic rings.

26. The enzymatic reactor cell of claim 24, wherein the nanowire comprises or uses maleimide.

27. The enzymatic reactor cell of claim 24, wherein the linker comprises a peptide or protein.

28. The enzymatic reactor cell of claim 27, wherein the peptide or protein comprises an amino acid sequence from Table 2 or SEQ ID NO: 176-180.

29. A method of performing an enzymatic reaction or enzymatic pathway with the use of an enzymatic reactor cell of claim 1 or a plurality of enzymatic reactor cells of claim 1.


SEQUENCE LISTING (181 sequences)

SEQ ID NO: 1 - protein, 42 aa, synthetic construct
MHGKTQATSGTIQSMHGKTQATSGTIQSMHGKTQATSGTIQS

SEQ ID NO: 2 - protein, 98 aa, synthetic construct
MHGKTQATSGTIQSMHGKTQATSGTIQSMHGKTQATSGTIQSMHGKTQATSGTIQSMHGKTQATSGTIQSMHGKTQATSGTIQSMHGKTQATSGTIQS

SEQ ID NO: 3 - protein, 12 aa, synthetic construct
WAGAKRLVLRRE

SEQ ID NO: 4 - protein, 10 aa, synthetic construct
HFSSWETQQG

SEQ ID NO: 5 - protein, 10 aa, synthetic construct
WYEKWQKANW

SEQ ID NO: 6 - protein, 8 aa, synthetic construct
VSGSSPDS

SEQ ID NO: 7 - protein, 36 aa, synthetic construct
SSKKSGSYSGSKGSRRILGGGGMHGKTQATSGTIQS

SEQ ID NO: 8 - protein, 30 aa, synthetic construct
MSPHPHPRHHHTGGGGMHGKTQATSGTIQS

SEQ ID NO: 9 - protein, 30 aa, synthetic construct
RGRRRRLSCRLLGGGGMHGKTQATSGTIQS

SEQ ID NO: 10 - protein, 30 aa, synthetic construct
DSARGFKKPGKRGGGGMHGKTQATSGTIQS

SEQ ID NO: 11 - protein, 30 aa, synthetic construct
HPPMNASHPHMHGGGGMHGKTQATSGTIQS

SEQ ID NO: 12 - protein, 30 aa, synthetic construct
HKDHHANQHVHMGGGGMHGKTQATSGTIQS

SEQ ID NO: 13 - protein, 16 aa, synthetic construct
HPPMNASHPHMHGGGG

SEQ ID NO: 14 - protein, 108 aa, synthetic construct
PTTGSCAVTYTANGWSGGFTAAVTLTNTGTTALSGWTLGFAFPSGQTLTQGWSARWAQSGSSVTATNEAWNAVLAPGASVEIGFSGTHTGTNTAPATFTVGGATCTTR

SEQ ID NO: 15 - protein, 108 aa, synthetic construct
SGPAGCQVLWGVNQWNTGFTANVTVKNTSSAPVDGWTLTFSFPSGQQVTQAWSSTVTQSGSAVTVRNAPWNGSIPAGGTAQFGFNGSHTGTNAAPTAFSLNGTPCTVG

SEQ ID NO: 16 - protein, 12 aa, synthetic construct
RAFIASRRIRRP

SEQ ID NO: 17 - protein, 8 aa, synthetic construct
RIIIRRIR

SEQ ID NO: 18 - protein, 12 aa, synthetic construct
RGRRRRLSCRLL

SEQ ID NO: 19 - protein, 18 aa, synthetic construct
SSKKSGSYSGSKGSRRIL

SEQ ID NO: 20 - protein, 12 aa, synthetic construct
MSPHPHPRHHHT

SEQ ID NO: 21 - protein, 12 aa, synthetic construct
DSARGFKKPGKR

SEQ ID NO: 22 - protein, 12 aa, synthetic construct
HPPMNASHPHMH

SEQ ID NO: 23 - protein, 12 aa, synthetic construct
HKDHHANQHVHM

SEQ ID NO: 24 - protein, 12 aa, synthetic construct
NPSSLFRYLPSD

SEQ ID NO: 25 - protein, 12 aa, synthetic construct
HSSYWYAFNNKT

SEQ ID NO: 26 - protein, 12 aa, synthetic construct
EAHVMHKVAPRP

SEQ ID NO: 27 - protein, 12 aa, synthetic construct
HHGHSPTSPQVR

SEQ ID NO: 28 - protein, 30 aa, synthetic construct
MVMPGDNIKMVVTLIHPIAMDDGLRFAIRE

SEQ ID NO: 29 - protein, 45 aa, synthetic construct
VGPNNVPYIVATITSNSAGGQPVSLANLKAMYSIAKKYDIPVVMD

SEQ ID NO: 30 - protein, 31 aa, synthetic construct
NNSWTRVAFAGLKFQDVGSFDYGRNYGVVYD

SEQ ID NO: 31 - protein, 120 aa, synthetic construct
QVQLVESGAEVKKPGESLKISCKGSGYSFPSYWINWVRQMPGKGLEWMGMIYPADSDTRYSPSFQGHVTISADKSINTAYLQWAGLKASDTAIYYCARLGIGGRYMSRWGQGTLVTVSSA

SEQ ID NO: 32 - protein, 10 aa, synthetic construct
LEYKRGYKPR

SEQ ID NO: 33 - protein, 20 aa, synthetic construct
DYKDDDDKAYSSGAPPMPPF

SEQ ID NO: 34 - protein, 61 aa, synthetic construct
FTLIELLIVVAIIGILAAIAIPQFSAYRVKAYNSAASSDLRNLKTALESAFADDQTYPPES

SEQ ID NO: 35 - protein, 144 aa, synthetic construct
FTLIELMIVVAIIGILAAIAIPQYQNYVARSEGASALASVNPLKTTVEEALSRGWSVKSGTGTEDATKKEVPLGVAADANKLGTIALKPDPADGTADITLTFTMGGAGPKNKGKIITLTRTAADGLWKCTSDQDEQFIPKGCSR

SEQ ID NO: 36 - protein, 167 aa, synthetic construct
MASNFKFKLLSQLKKRAEGGFTLIELLVVVIIIGVLAAIALPNLLGQVGKARESEAKSTIGALNRAQQGYFTEKGTFATDTETLEVPAPDGNFFSFAVNTADNTEAIQDATALNWEADGTRSMSGGTFYDSGTRAFSTVVCRAEAGSEDTPPTPGGANDCGGAEVIK

SEQ ID NO: 37 - protein, 28 aa, synthetic construct
ELKAIAQEFKAIAKEFAIAFEFKAIAQK

SEQ ID NO: 38 - protein, 7 aa, synthetic construct
YYACAYY

SEQ ID NO: 39 - protein, 7 aa, synthetic construct
GNNQQNY

SEQ ID NO: 40 - protein, 9 aa, synthetic construct
KVQIINKKL

SEQ ID NO: 41 - protein, 5 aa, synthetic construct
VGGLG

SEQ ID NO: 42 - protein, 8 aa, synthetic construct
VGGLGHHH

SEQ ID NO: 43 - protein, 8 aa, synthetic construct
VGGLGWWW

SEQ ID NO: 44 - protein, 8 aa, synthetic construct
VGGLGYYY

SEQ ID NO: 45 - protein, 7 aa, synthetic construct
VGGLFFF

SEQ ID NO: 46 - protein, 10 aa, synthetic construct
SVNVTQVGFP

SEQ ID NO: 47 - protein, 13 aa, synthetic construct
SVNVTQVGFPHHH

SEQ ID NO: 48 - protein, 13 aa, synthetic construct
SVNVTQVGFPWWW

SEQ ID NO: 49 - protein, 13 aa, synthetic construct
SVNVTQVGFPYYY

SEQ ID NO: 50 - protein, 6 aa, synthetic construct
RKLPDA

SEQ ID NO: 51 - protein, 9 aa, synthetic construct
PPPYPPPWC

SEQ ID NO: 52-53 - skipped (2 entries)

SEQ ID NO: 54 - protein, 7 aa, synthetic construct
EPLQLKM

SEQ ID NO: 55 - protein, 12 aa, synthetic construct
GAMHLPWHMGTL

SEQ ID NO: 56 - protein, 12 aa, synthetic construct
IMVTASSAYRRY

SEQ ID NO: 57 - protein, 16 aa, synthetic construct
VIAGASLWWSEKLVIA

SEQ ID NO: 58 - protein, 13 aa, synthetic construct
CALNNDEVDKFAM

SEQ ID NO: 59 - protein, 13 aa, synthetic construct
KKNYSSSISSIHC

SEQ ID NO: 60 - protein, 15 aa, synthetic construct
CGGHSSKLQFWYFWY

SEQ ID NO: 61 - protein, 12 aa, synthetic construct
HWSAWWIRSNQS

SEQ ID NO: 62 - protein, 12 aa, synthetic construct
IMVTESSDYSSY

SEQ ID NO: 63 - protein, 12 aa, synthetic construct
IMVTASSAYDDY

SEQ ID NO: 64 - protein, 12 aa, synthetic construct
IMVTQSSNYSSY

SEQ ID NO: 65 - protein, 12 aa, synthetic construct
IMVTKSSDYSSY

SEQ ID NO: 66 - protein, 12 aa, synthetic construct
HSSAAAAFNNKT

SEQ ID NO: 67 - protein, 12 aa, synthetic construct
HTSYWYAFNTKT

SEQ ID NO: 68 - protein, 12 aa, synthetic construct
YTTHVLPFAPSS

SEQ ID NO: 69 - protein, 11 aa, synthetic construct
HAWVDWIRPIH

SEQ ID NO: 70 - protein, 12 aa, synthetic construct
HWKHPWGAWDTL

SEQ ID NO: 71 - protein, 7 aa, synthetic construct
QQQLSTH

SEQ ID NO: 72 - protein, 18 aa, synthetic construct
GGPDSARGFKKPGKRGPC

SEQ ID NO: 73 - protein, 15 aa, synthetic construct
GGGMHPNAGHGSLMR

SEQ ID NO: 74 - protein, 36 aa, synthetic construct
TPQSHYGQCGGGYSGPTVCASGTTCQVLNPYYSQCL

SEQ ID NO: 75 - protein, 157 aa, synthetic construct
GVVSVQFNNGSSPASSNSIYARFKVTNTSGSPINLADLKLRYYYTQDADKPLTFWCDHAGYMSGSNYIDATSKVTGSFKAVSPAVTNADHYLEVALNSDAGSLPAGGSIEIQTRFARNDWSNFDQSNDWSYTAAGSYMDWQKISAFVGGTLAYGSTP

SEQ ID NO: 76 - protein, 12 aa, synthetic construct
QPYLFATDSLIK

SEQ ID NO: 77 - protein, 12 aa, synthetic construct
GHTHYHAVRTQT

SEQ ID NO: 78 - protein, 9 aa, synthetic construct
RLLLRRLRR

SEQ ID NO: 79 - protein, 13 aa, synthetic construct
KRAFIASRRIRRP

SEQ ID NO: 80 - skipped

SEQ ID NO: 81 - protein, 12 aa, synthetic construct
HGGGHGHGGGHG

SEQ ID NO: 82 - protein, 12 aa, synthetic construct
TSNAVHPTLRHL

SEQ ID NO: 83 - protein, 12 aa, synthetic construct
NFMSLPRLGHMH

SEQ ID NO: 84 - protein, 9 aa, synthetic construct
AHHAHHAAD

SEQ ID NO: 85 - protein, 7 aa, synthetic construct
SGTGASY

SEQ ID NO: 86 - protein, 8 aa, synthetic construct
DAHKSEVA

SEQ ID NO: 87 - protein, 12 aa, synthetic construct
VPSSGPQDTRTT

SEQ ID NO: 88 - protein, 12 aa, synthetic construct
YSPDPRPWSSRY

SEQ ID NO: 89 - protein, 6 aa, synthetic construct
SYHHHH

SEQ ID NO: 90 - protein, 12 aa, synthetic construct
ERSWTLDSALSM

SEQ ID NO: 91 - protein, 12 aa, synthetic construct
SNNDLSPLQTSH

SEQ ID NO: 92 - protein, 12 aa, synthetic construct
DSSNPIFWRPSS

SEQ ID NO: 93 - protein, 12 aa, synthetic construct
SILSTMSPHGAT

SEQ ID NO: 94 - protein, 12 aa, synthetic construct
SHALPLTWSTAA

SEQ ID NO: 95 - protein, 12 aa, synthetic construct
HVSIHRTTHHEM

SEQ ID NO: 96 - protein, 12 aa, synthetic construct
MKPDKAIRLDLL

SEQ ID NO: 97 - protein, 12 aa, synthetic construct
HYPTAKFHAERL

SEQ ID NO: 98 - protein, 12 aa, synthetic construct
TKNMLSLPVGPG

SEQ ID NO: 99 - protein, 12 aa, synthetic construct
FNTGSQMHQKFP

SEQ ID NO: 100 - protein, 12 aa, synthetic construct
HHTHRVDVHQTR

SEQ ID NO: 101 - protein, 12 aa, synthetic construct
FGLTAPRSASIL

SEQ ID NO: 102 - protein, 12 aa, synthetic construct
APRLPQSLLPQL

SEQ ID NO: 103 - protein, 12 aa, synthetic construct
ATIHDAFYSAPE

SEQ ID NO: 104 - protein, 12 aa, synthetic construct
NLNPNTASAMHV

SEQ ID NO: 105 - protein, 12 aa, synthetic construct
NLTIASYPSMVV

SEQ ID NO: 106 - protein, 12 aa, synthetic construct
QSHYRHISPAQV

SEQ ID NO: 107 - protein, 12 aa, synthetic construct
QMDISLGRWSSM

SEQ ID NO: 108 - protein, 12 aa, synthetic construct
YMKQIPAGRTNP

SEQ ID NO: 109 - protein, 12 aa, synthetic construct
VQHNTKYSVVIR

SEQ ID NO: 110 - skipped

SEQ ID NO: 111 - protein, 12 aa, synthetic construct
VQHNTKYSVVIR

SEQ ID NO: 112 - protein, 12 aa, synthetic construct
MTWDPSLASPRS

SEQ ID NO: 113 - protein, 12 aa, synthetic construct
YQLRPNAESLRF

SEQ ID NO: 114 - protein, 17 aa, synthetic construct
KCTSDQDEQFIPKGCSK

SEQ ID NO: 115 - protein, 150 aa, synthetic construct
FTLIELMIVVAIIGILAAIAIPAYQDYTARAQLSEAMTLASGLKTKVSDIFSQDGSCPANTAATAGIEKDTDINGKYVAKVTTGGTAAASGGCTIVATMKASDVATPLRGKTLTLTLGNADKGSYTWACTSNADNKYLPKTCQTATTTTP

SEQ ID NO: 116 - protein, 17 aa, synthetic construct
  SITE 1: note = N-terminus is acetylated
  SITE 17: note = C-terminus is amidated
ACTSNADNKYLPKTCQT

SEQ ID NO: 117 - protein, 17 aa, synthetic construct
  SITE 1: note = N-terminus is acetylated
  SITE 17: note = C-terminus is carboxylated
ACTSNADNKYLPKTCQT

SEQ ID NO: 118 - protein, 17 aa, synthetic construct
  SITE 1: note = N-terminus is acetylated
  SITE 17: note = C-terminus is carboxylated
ACISTQDPMFTPKGCDN

SEQ ID NO: 119 - protein, 17 aa, synthetic construct
ACKSTQDPMFTPKGCDN

SEQ ID NO: 120 - protein, 9 aa, synthetic construct
RRTVKHHVN

SEQ ID NO: 121 - protein, 12 aa, synthetic construct
QMDTSTSLAPSR

SEQ ID NO: 122 - protein, 9 aa, synthetic construct
AHHAHHAAD

SEQ ID NO: 123 - protein, 9 aa, synthetic construct
CHKKPSKSC

SEQ ID NO: 124 - protein, 16 aa, synthetic construct
AGSWLRDIWTWLQSAL

SEQ ID NO: 125 - protein, 7 aa, synthetic construct
LPPWKLK

SEQ ID NO: 126 - protein, 12 aa, synthetic construct
THRTSTLDYFVI

SEQ ID NO: 127 - protein, 12 aa, synthetic construct
TSDIKSRSPHHR

SEQ ID NO: 128 - protein, 12 aa, synthetic construct
HTQNMRMYEPWF

SEQ ID NO: 129 - protein, 47 aa, synthetic construct
AIKLVQSPNGNFAASFVLDGTKWIFKSKYYDSSKGYWVGIYEVWDRK

SEQ ID NO: 130 - protein, 47 aa, synthetic construct
AIKLVQSPNGNFAASFVLDGTKWTFKSKHYDSSKGYWVGIYKVWDRK

SEQ ID NO: 131 - protein, 47 aa, synthetic construct
AIKLVQSPNGNFAASFVLDGTKWIFKSKRYDSSKGYWVGIYEVWRRK

SEQ ID NO: 132 - DNA, 13 bp, synthetic construct
  source 1..13: mol_type = other DNA
gcaactaggctcg

SEQ ID NO: 133 - DNA, 13 bp, synthetic construct
  source 1..13: mol_type = other DNA
cgagcctagttgc

SEQ ID NO: 134 - DNA, 22 bp, synthetic construct
  source 1..22: mol_type = other DNA
gcacctgaaccgcatggactcg

SEQ ID NO: 135 - DNA, 22 bp, synthetic construct
  source 1..22: mol_type = other DNA
cgagtccatgcggttcaggtgc

SEQ ID NO: 136 - skipped

SEQ ID NO: 137 - protein, 8 aa, synthetic construct
VDGGGGGS

SEQ ID NO: 138 - protein, 14 aa, synthetic construct
GTGGGGVDGGGGGS

SEQ ID NO: 139 - protein, 4 aa, synthetic construct
GGGG

SEQ ID NO: 140 - protein, 5 aa, synthetic construct
GGGGG

SEQ ID NO: 141 - protein, 6 aa, synthetic construct
GGGGGG

SEQ ID NO: 142 - protein, 8 aa, synthetic construct
GGGGGGGG

SEQ ID NO: 143 - skipped

SEQ ID NO: 144 - protein, 4 aa, synthetic construct
GGGS

SEQ ID NO: 145 - protein, 5 aa, synthetic construct
GGGGS

SEQ ID NO: 146 - protein, 5 aa, synthetic construct
PAPAP

SEQ ID NO: 147 - protein, 5 aa, synthetic construct
EAAAK

SEQ ID NO: 148 - protein, 5 aa, synthetic construct
GGYGG

SEQ ID NO: 149 - protein, 5 aa, synthetic construct
GGFGG

SEQ ID NO: 150 - protein, 6 aa, synthetic construct
GGFFGG

SEQ ID NO: 151 - protein, 6 aa, synthetic construct
GGYYGG

SEQ ID NO: 152 - protein, 8 aa, synthetic construct
GGSYWGGS

SEQ ID NO: 153 - protein, 14 aa, synthetic construct
GGGGSEAAAKGGGS

SEQ ID NO: 154 - protein, 19 aa, synthetic construct
GGGGSEAAAKEAAAKGGGS

SEQ ID NO: 155 - protein, 205 aa, Thermus thermophilus
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVKAILGLPSHAAIPALVALGYPAEEGYPSHRLPLERVVLWR

SEQ ID NO: 156 - protein, 217 aa, synthetic construct
MNLNPNTASAMHVEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVKAILGLPSHAAIPALVALGYPAEEGYPSHRLPLERVVLWR

SEQ ID NO: 157 - protein, 217 aa, synthetic construct
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVKAILGLPSHAAIPALVALGYPAEEGYPSHRLPLERVVLWRNLNPNTASAMHV

SEQ ID NO: 158 - protein, 219 aa, synthetic construct
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVKAILGLPSHAAIPALVALGYPAEEGYPSHRLPLERVVLWRGSHSSYWYAFNNKT

SEQ ID NO: 159 - protein, 205 aa, synthetic construct
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVKAILGLPSHAAIPALVALGYPAEEGYPSYRLPLERVVLWR

SEQ ID NO: 160 - protein, 205 aa, synthetic construct
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVKAILGLPSRAAIPALVALGYPAEEGYPSYRLPLERVVLWR

SEQ ID NO: 161 - protein, 205 aa, synthetic construct; identical to SEQ ID NO: 159

SEQ ID NO: 162 - protein, 205 aa, synthetic construct
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVRAILGLPSHAAIPALVALGYPAEEGYPSHRLPLERVVLWR

SEQ ID NO: 163 - protein, 205 aa, synthetic construct
  VARIANT 166: note = K or R
  VARIANT 174: note = H or R
  VARIANT 194: note = H or Y
MEATLPVLDAKTAALKRRSIRRYRKDPVPEGLLREILEAALRAPSAWNLQPWRIVVVRDPATKRALREAAFGQAHVEEAPVVLVLYADLEDALAHLDEVIHPGVQGERREAQKQAIQRAFAAMGQEARKAWASGQSYILLGYLLLLLEAYGLGSVPMLGFDPERVXAILGLPSXAAIPALVALGYPAEEGYPSXRLPLERVVLWR

SEQ ID NO: 164 - protein, 205 aa, synthetic construct; identical to SEQ ID NO: 163
  VARIANT 166: note = K, R, H, F, Y or W
  VARIANT 174: note = K, R, H, F, Y or W
  VARIANT 194: note = K, R, H, F, Y or W

SEQ ID NO: 165 - protein, 509 aa, Amphibacillus xylanus
MLDKDIKQQLEQYLALLENDIVIKVSVGDDKVSKDTLELVNEIADMSSMISVEETTLERTPSFSINRKGEPDSGVVFAGIPLGHEFTSLVLALLQVSGRAPKVEASVIDAIKKVEGKHDFVTYVSLTCQNCPDVVQALNLMSVVNPNITHTMVEGSAFKDEVDRLNILAVPSVYLNGEFMASGRMTIEDILGHLGSGIDASELNEKDPFDVLVIGGGPAGASSAIYAARKGIRVGIIADRFGGQVMDTLGIENFIGTKYTEGPKLISEIEQHVNEYNIDVMKGLRAHNIEKKDLFEVQLDNGAVLKSKTIILATGARWRDIGVPGEKEYKNKGVAYCTHCDAPLFEGKHVAVVGGGNSGIESALDLAGIVKHVTVFEFMSELKADAVLQERLRSLPNVDVILNAQTTEITGDETVKGISYIDRTTNEEKHVELQGAFIQIGLAPNTEWLGDTVERNQIGEIVIDKKGQTSVPGIFAAGDCTDTPYKQIIVSMGAGATAALGAFDYLLRN

SEQ ID NO: 166 - protein, 632 aa, Archaeoglobus fulgidus
MKLFEPIEFGGMKVKNRIVMPAAELNYHTPDGRPTDRLLRFYEERAKGGIGFAVVGIAKIEPHFFGGIAAHSDEFIPDLKKIADVFHKYDVKCALQLWHPGRYEISFDPNRQPVAPSPIPPPIFTKRTPKELTKEEILQIEEEFADAAVRAKKAGFDAVELIGSAGYLISQFFSPATNKRTDEYGGSLENRTRFAVEIIQKIKEKCGESYPVMIRIPGDEFIEGGNTVREMKQIAKILEDAGVVAINVMAGWHESRKPLTTMLVPRGGFAYLAAEIKSAVSVPVIASHRINDPIVAEKILQEGKADMVAMLRALIADPELPKKAKEGRFDEIRYCVACNQGCMDMVMQAQPVTCLVNPIVGREAEFENLKAEKPKKVVIVGGGPGGCMAAEMLARKGHKVVLFEKTDKLGGQLNLAAKSPLGYEFAEVGKYFMNVLPKLGVEVRYNTEADAGKVLAENPDVAVIAVGASPLIPPIPGVENAVTAFDVLAGRAEVGNSVVVIGGGGVGCDVAAELANEGKKVTIVEMLPKIGQDIGISTRWTVLMYLKEKGVEMLTNTKAVEIKPNAVVVEQNGERKELQCDTAVIAVGTKPNNGLYDELQGKVSELYKIGDCVKPRKALDATREGADLALKV

SEQ ID NO: 167 - protein, 249 aa, Bacillus subtilis
MNNTIETILNHRSIRSFTDQLLTAEEIDTLVKSAQAASTSSYVQAYSIIGVSDPEKKRELSVLAGNQPYVEKNGHFFVFCADLYRHQQLAEEKGEHISELLENTEMFMVSLIDAALAAQNMSIAAESMGLGICYIGGIRNELDKVTEVLQTPDHVLPLFGLAVGHPANLSGKKPRLPKQAVYHENTYNVNTDDFRHTMNTYDKTISDYYRERTNGKREETWSDQILNFMKQKPRTYLNDYVKEKGFNKN

SEQ ID NO: 168 - protein, 448 aa, Lactococcus lactis
MRAIIIGSGAAGLTTASTIRKYNKDMEIVVITKEKEIAYSPCAIPYVIEGAIKSFDDIIMHTPEDYKRERNIDILTETTVIDVDSKNNKIKCVDKDGNEFEMNYDYLVLATGAEPFIPPIEGKDLDGVFKVRTIEDGRAILKYIEENGCKKVAVVGAGAIGLEMAYGLKCRGLDVLVVEMAPQVLPRFLDPDMAEIVQKYLEKEGIKVMLSKPLEKIVGKEKVEAVYVDGKLYDVDMVIMATGVRPNIELAKKAGCKIGKFAIEVNEKMQTSIPNIYAVGDCVEVIDFITGEKTLSPFGTAAVRQGKVAGKNIAGVEAKFYPVLNSAVSKIGDLEIGGTGLTAFSANLKRIPIVIGRAKALTRARYYPGGKEIEIKMIFNEDGKVVGCQIVGGERVAERIDAMSIAIFKKVSAEELANMEFCYAPPVSMVHEPLSLAAEDALKKLSNK

SEQ ID NO: 169 - protein, 245 aa, Priestia megaterium
MNEAIRTIQDHRSIRQYTDEAVSDEHLDTIIQSAQSAASSINGQQVTIISVQDKEKKKKLSELAGNQAWIDQAPLFLIFCADFNRAKIAAELNDAPLGVTDGLESILVGATDAGISLEAATVAAESLGLGTVPIGGIRRKPLEVIELLDLPEYVFPVSGLVVGHPSDHSAKKPRLPQAAVHHRESYNHDLKSLIQDYDAEMAEYMKKRTNGADDRNWSQTVSAIYKTIYYPEVRAMLEKQGFKFE

SEQ ID NO: 170 - protein, 438 aa, Pyrococcus furiosus
MRIVVIGSGTAGSNFALFMRKLDRKAEITVIGKEETMQYSPCALPHVISGVIEKPEDVIVFPNEFYEKQRIKLLLNTEAKKIDRERKVVVTDKGEIPYDKLVIATGSKAFVPPIKGVENEGVFTLKSLEDVRKIKEFIKKRNPKNAVVIGAGLIGLEGAEAFAKLGMKVTVVELLEHLLPTMLDKDIAKIVEENMRKYGVDFKFGVGVDEIIGDPVEKVKVGEEEIDADIVLVATGVRANVELAKEAGLEVNRGIVVNEYLQTSDPDIYAIGDCAEVIDAVTGKRTLSQLGTSAVRMAKVAAENIAGRNVKFRPVFNTAITEIFDLEIGAFGITEERAKKEEIEVVVGKFRGSTKPEYYPGGKPIVVKLIFRKEDRRLIGAQIVGGERVWGRIMTLSALAQKGATVEDVVYLETAYAPPISPTIDPITIAAEMAMRKL

SEQ ID NO: 171 - protein, 510 aa, Streptococcus mutans
  source 1..510: note = Streptococcus mutans JC2
MALDAEIKEQLGQYLQLLECEIVLQAQLKDDANSQKVKEFLQEIVAMSPMISLDEKELPRTPSFRIAKKGQESGVEFAGLPLGHEFYFVYLGSVTGFRASAKVETDIVKRIQAVDEPMHFETYVSLTCHNCPDVVQAFNIMSVVNPNISHTMVEGGMFKDEIEAKGIMSVPTVYKDGTEFTSGRASIEQLLDLIAGPLKEDAFDDKGVFDVLVIGGGPAGNSAAIYAARKGVKTGLLAETMGGQVMETVGIENMIGTPYVEGPQLMAQVEEHTKSYSVDIMKAPRAKSIQKTDLVEVELDNGAHLKAKTAVLALGAKWRKINVPGEKEFFNKGVTYCPHCDGPLFTDKKVAVIGGGNSGLEAAIDLAGLASHVYILEFLPELKADKILQDRAEALDNITILTNVATKEIIGNDHVEGLRYSDRTTNEEYLLDLEGVFVQIGLVPSTDWLKDSGLALNEKGEIIVAKDGATNIPAIFAAGDCTDSAYKQIIISMGSGATAALGAFDYLIRN

SEQ ID NO: 172 - protein, 347 aa, Sus scrofa
MRVVVIGAGVIGLSTALCIHERYHSVLQPLDVKVYADRFTPFTTTDVAAGLWQPYTSEPSNPQEANWNQQTFNYLLSHIGSPNAANMGLTPVSGYNLFREAVPDPYWKDMVLGFRKLTPRELDMFPDYRYGWFNTSLILEGRKYLQWLTERLTERGVKFFLRKVESFEEVARGGADVIINCTGVWAGVLQPDPLLQPGRGQIIKVDAPWLKNFIITHDLERGIYNSPYIIPGLQAVTLGGTFQVGNWNEINNIQDHNTIWEGCCRLEPTLKDAKIVGEYTGFRPVRPQVRLEREQLRFGSSNTEVIHNYGHGGYGLTIHWGCALEVAKLFGKVLEERNLLTMPPSHL

SEQ ID NO: 173 - protein, 439 aa, Thermococcus kodakarensis
  source 1..439: note = Thermococcus kodakarensis KOD1
MKIVVVGSGTAGSNFALFMRKLDRKAEITVIGKEPTMQYSPCALPHVVSGTIEKPEDIIVFPNEFYEKQKINLMLNTEAKAIDRERKVVVTDKGEVPYDKLVLAVGSKAFIPPIKGVENEGVFTLKSLDDVRRIKAYIAERKPKKAVVIGAGLIGLEGAEAFAKLGMEVLIVELMDRLMPTMLDKDTAKLVQAEMEKYGVSFRFGVGVSEIIGSPVRAVKIGDEEVPADLVLVATGVRANTDLAKQAGLEVNRGIVVNEHLQTSDPEVYAIGDCAEVIDAVTGKRTLSQLGTSAVRMAKVAAEHIAGKDVSFRPVFNTAITELFGLEIGTFGITEERAKKEDIEVAVGKFKGSTKPEYYPGGKPITVKLIFRKSDRKLIGGQIVGGERVWGRIMTLSALAQKGATVEDVAYLETAYAPPISPTIDPITVAAEMAQRKLR

SEQ ID NO: 174 - protein, 447 aa, Thermococcus kodakarensis
  source 1..447: note = Thermococcus kodakarensis KOD1
MKYDVVVIGGSAGGLTAAISAKRFYPDKSVLVIKKEDVSMIPCGIPYIFGTLRSVEDDVLPTERFLKPLGIDVLVDEVTEINPKSKTLLTKSGREIGWEKLVLATGSRPQIPDIPGVELEGVYTVSKDYHYLKELKKRVEDAEKVVIIGGGFIALEVGDEIRKLGKDVTIVVRSRLLRNSFDPEFSEMIENRLKEVGINVVYGHVERLVGRERVEGVKLVEGGEIPADLVILSTGYRPNVELAVKTGLKVTRYGIWTDEYMRTSCPDIFAVGDCVEHRDFFTGKPFPLMLASTATFEARIAGANLFKLQIVRENRRTIGAYSTHVAGLTLAAAGLTEEAAKREGFEVIVGRATGPDRHPAKFEDTSMVTVKLIFSRDRGAILGAQLAGGKSVGEMINVLALAIQKRLTASELYTLQIATHPLLTASPVGYQILQAAEDALAKLRAGA

SEQ ID NO: 175 - protein, 205 aa, Thermus thermophilus; identical to SEQ ID NO: 162
  source 1..205: note = Thermus thermophilus HB8

SEQ ID NO: 176 - protein, 16 aa, synthetic construct
  REPEAT 1..2: note = single repeat of GS
  VARIANT 3..16: note = up to 7 copies of GS can be deleted
GSGSGSGSGSGSGSGS

SEQ ID NO: 177 - protein, 15 aa, synthetic construct
  REPEAT 1..3: note = single repeat of GGS
  VARIANT 4..15: note = up to 4 copies of GGS can be deleted
GGSGGSGGSGGSGGS

SEQ ID NO: 178 - protein, 16 aa, synthetic construct
  REPEAT 1..4: note = single repeat of GGGS
  VARIANT 5..16: note = up to 3 copies of GGGS can be deleted
GGGSGGGSGGGSGGGS

SEQ ID NO: 179 - protein, 20 aa, synthetic construct
  REPEAT 1..5: note = single repeat of GGGGS
  VARIANT 6..20: note = up to 3 copies of GGGGS can be deleted
GGGGSGGGGSGGGGSGGGGS

SEQ ID NO: 180 - protein, 20 aa, synthetic construct
  REPEAT 1..5: note = single repeat of EAAAK
  VARIANT 6..20: note = up to 3 copies of EAAAK can be deleted
EAAAKEAAAKEAAAKEAAAK

SEQ ID NO: 181 - protein, 448 aa, Methanocaldococcus jannaschii; identical to SEQ ID NO: 168